(19) United States
(12) Patent Application Publication
(10) Pub. No.: US 2024/0067716 A1
(43) Pub. Date: Feb. 29, 2024

(54) IONIC LIQUID FORMULATIONS FOR TREATING INFLAMMATORY AND AUTOIMMUNE DISEASES

(71) Applicant: i2O Therapeutics, Inc., Cambridge, MA (US)

(72) Inventors: Tyler BROWN, Cambridge, MA (US); Kelly IBSEN, Boston, MA (US)

(21) Appl. No.: 18/501,735

(22) Filed: Nov. 3, 2023

Related U.S. Application Data
(63) Continuation of application No. PCT/US2022/027794, filed on May 5, 2022.
(60) Provisional application No. 63/184,333, filed on May 5, 2021.

(51) Int. Cl. C07K 16/24; A61K 47/18

(52) U.S. Cl. CPC C07K 16/241; A61K 47/186; C07K 2317/24; C07K 2317/94

(57) ABSTRACT

Disclosed herein are compositions comprising ionic liquids and the use thereof for treating conditions or diseases in a subject.

### CROSS-REFERENCE

This application is a continuation of International Application No. PCT/US2022/027794 filed May 5, 2022, which claims the benefit of U.S. Provisional Patent Application No. 63/184,333 filed May 5, 2021, the entire contents of which are hereby incorporated by reference.

### BACKGROUND OF THE INVENTION

Inflammatory and autoimmune diseases are diseases characterized by a hyperactive immune system. Despite advances, there remains a need for novel treatments for inflammatory and autoimmune diseases.

### SUMMARY OF THE INVENTION

Provided herein, in some aspects, inter alia, is a composition comprising a therapeutic agent and at least one ionic liquid, wherein the therapeutic agent comprises an antibody or an antibody reagent.

In some embodiments, the at least one ionic liquid comprises choline as a cation component.

In some embodiments, the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the at least one ionic liquid comprises a cationic component and an anionic component in the molar ratio of 1;1, 1:2, 1:3, 1:4, 2:1, 3:1, or 4:1.

In some embodiments, the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

In some embodiments, the composition comprises a first ionic liquid and a second ionic liquid, wherein the first ionic liquid and the second ionic liquid are different.

In some embodiments, the first ionic liquid comprises choline as a cation component, the second ionic liquid comprises choline as a cation component, or a combination thereof.

In some embodiments, the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the first ionic liquid and the second ionic liquid comprise: (i) choline-3-phenylpropanoic acid in the molar ratio of 1:2 and choline-glycolic acid in the molar ratio of 2:1, respectively; (ii) choline-3-phenylpropanoic acid in the molar ratio of 1:1 and choline-glycolic acid in the molar ratio of 2:1, respectively; or (iii) choline-3-phenylpropanoic acid in the molar ratio of 1:1 and choline-glycolic acid in the molar ratio of 1:1, respectively.

In some embodiments, the composition as provided herein further comprises glycerol.

In some embodiments, the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

In some embodiments, the composition comprises at least one ionic liquid and glycerol in the ratio of: (i) 10%:90%, wherein the at least one ionic liquid comprises choline-3-phenylpropanoic acid in the molar ratio of 1:1; (ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1; (iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1; (iv) 75%:25%, wherein the at least one ionic liquid comprises choline-tartaric acid in the molar ratio of 2:1 or choline-citric acid in the molar ratio of 3:1; or (v) 90%:10%, wherein the at least one ionic liquid comprises choline-tartaric acid in the molar ratio of 2:1.

In some embodiments, the composition comprises the first ionic liquid and the second ionic liquid, and wherein the composition comprises the first ionic liquid, the second ionic liquid, and glycerol in the ratio of: (i) 10%:45%:45%, wherein the first ionic liquid comprises choline-3-phenylpropanoic acid in the molar ratio of 1:2 and the second ionic liquid comprises choline-glycolic acid in the molar ratio of 2:1; or wherein the first ionic liquid comprises choline-3-phenylpropanoic acid in the molar ratio of 1:1 and the second ionic liquid comprises choline-glycolic acid in the molar ratio of 2:1; or (ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

In some embodiments, the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab- adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

In some embodiments, the antibody or the antibody reagent is infliximab or a biosimilar thereof, adalimumab or a biosimilar thereof, or a combination thereof.

In some embodiments, the infliximab or a biosimilar thereof is selected from the group consisting of infliximab, infliximab-dyyb, infliximab-abda, infliximab-qbtx, and infliximab-axxq.

In some embodiments, the adalimumab or a biosimilar thereof is selected from the group consisting of adalimumab, adalimumab-atto, adalimumab-adaz, adalimumab-afzb, and adalimumab-bwwd.

In some embodiments, the composition as provided herein further comprises one or more additional agents.

In some embodiments, the one or more additional agents are selected from the group consisting of a nucleic acid, a small molecule, and a polypeptide.

In some embodiments, the one or more additional agents are selected from the group consisting of a corticosteroid, an aminosalicylates, an immunomodulator, a monoclonal antibody, and a biologic.

In some embodiments, the composition is formulated for delivery to a mucus membrane, across a mucus membrane, or a combination thereof.

In some embodiments, the mucus membrane is a nasal membrane, an oral membrane, a vaginal membrane, or a combination thereof.

In some embodiments, the composition is formulated for administered to a gastrointestinal tract.

In some embodiments, the composition is formulated for subcutaneous administration, intravenous administration, topical administration, intrajejunal administration, oral administration, or a combination thereof. In another embodiment, the composition is formulated for intracolonic administration or administration to the colon.

In some embodiments, the composition is formulated for oral administration.

In some embodiments, the composition is formulated in a form selected from the group consisting of a tablet, a pill, a caplet, a capsule, a spray, an aerosol, a syrup, a liquid, and a combination thereof.

In some embodiments, the composition is encapsulated in a capsule.

In some aspects, provided herein is a pharmaceutical composition comprising the composition as provided herein, and a pharmaceutically acceptable excipient.

In some aspects, provided herein is a method of treating a disease or a disorder in a subject in need thereof, comprising administering to the subject a therapeutically effective amount of the composition as provided herein or the pharmaceutical composition as provided herein.

In some embodiments, the disease or the disorder is an inflammatory disease, an autoimmune disease, or a combination thereof.

In some embodiments, the inflammatory disease, the autoimmune disease, or a combination thereof is characterized by a hyperactive immune system.

In some embodiments, the inflammatory disease, the autoimmune disease, or a combination thereof is selected from the group consisting of rheumatoid arthritis, Crohn's disease, ulcerative colitis, psoriasis, psoriatic arthritis, ankylosing spondylitis, inflammatory bowel disease, and a combination thereof.

In some embodiments, the disease or the disorder is diabetes.

In a further embodiment, the disease or disorder is obesity. In yet another embodiment, is a method for treating a metabolic disease including obesity, type 2 diabetes melliuts, non-alcoholic fatty liver disease and diseases in which GDF15 is expressed.

In some embodiments, the method as provided herein further comprises administering one or more additional agents.

In some embodiments, the one or more additional agents are selected from the group consisting of a nucleic acid, a small molecule, and a polypeptide.

In some embodiments, the one or more additional agents are selected from the group consisting of a corticosteroid, an aminosalicylates, an immunomodulator, a monoclonal antibody, and a biologic.

In some embodiments, the method delivers the composition to a mucus membrane, across a mucus membrane, or a combination thereof in the subject.

In some embodiments, the mucus membrane is a nasal membrane, an oral membrane, a vaginal membrane, or a combination thereof.

In some embodiments, the administering comprises administering to a gastrointestinal tract in the subject.

In some embodiments, the administering comprises subcutaneous administration, intravenous administration, topical administration, intrajejunal administration, oral administration, or a combination thereof. In another embodiment, the composition is formulated for intracolonic administration or administration to the colon.

In some embodiments, the administering comprises oral administration.

In some embodiments, the composition is administered in a single dose.

In some embodiments, the composition is administered in multiple doses.

In some aspects, provided herein is a method of increasing the solubility of a therapeutic agent comprising formulating a composition comprising the therapeutic agent and at least one ionic liquid, wherein the therapeutic agent comprises an antibody or an antibody reagent.

In some embodiments, the at least one ionic liquid comprises choline as a cation component.

In some embodiments, the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the at least one ionic liquid comprises a cationic component and an anionic component in the molar ratio of 1;1, 1:2, 1:3, 1:4, 2:1, 3:1, or 4:1.

In some embodiments, the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

In some embodiments, the composition comprises a first ionic liquid and a second ionic liquid, wherein the first ionic liquid and the second ionic liquid are different.

In some embodiments, the first ionic liquid comprises choline as a cation component, the second ionic liquid comprises choline as a cation component, or a combination thereof.

In some embodiments, the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the first ionic liquid and the second ionic liquid comprise: (i) choline-3-phenylpropanoic acid in the molar ratio of 1:2 and choline-glycolic acid in the molar ratio of 2:1, respectively; (ii) choline-3-phenylpropanoic acid in the molar ratio of 1:1 and choline-glycolic acid in the molar ratio of 2:1, respectively; or (iii) choline-3-phenylpropanoic acid in the molar ratio of 1:1 and choline-glycolic acid in the molar ratio of 1:1, respectively.

In some embodiments, the method as provided herein further comprises glycerol.

In some embodiments, the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

In some embodiments, the composition comprises at least one ionic liquid and glycerol in the ratio of: (i) 10%:90%, wherein the at least one ionic liquid comprises choline-3-phenylpropanoic acid in the molar ratio of 1:1; (ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1; (iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1; (iv) 75%:25%, wherein the at least one ionic liquid comprises choline-tartaric acid in the molar ratio of 2:1 or choline-citric acid in the molar ratio of 3:1; or (v) 90%:10%, wherein the at least one ionic liquid comprises choline-tartaric acid in the molar ratio of 2:1.

In some embodiments, the composition comprises the first ionic liquid and the second ionic liquid, and wherein the composition comprises the first ionic liquid, the second ionic liquid, and glycerol in the ratio of: (i) 10%:45%:45%, wherein the first ionic liquid comprises choline-3-phenylpropanoic acid in the molar ratio of 1:2 and the second ionic liquid comprises choline-glycolic acid in the molar ratio of 2:1; or wherein the first ionic liquid comprises choline-3-phenylpropanoic acid in the molar ratio of 1:1 and the second ionic liquid comprises choline-glycolic acid in the molar ratio of 2:1; or (ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

In some embodiments, the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab-adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

In some embodiments, the antibody or the antibody reagent is infliximab or a biosimilar thereof, adalimumab or a biosimilar thereof, or a combination thereof.

In some embodiments, the infliximab or a biosimilar thereof is selected from the group consisting of infliximab, infliximab-dyyb, infliximab-abda, infliximab-qbtx, and infliximab-axxq.

In some embodiments, the adalimumab or a biosimilar thereof is selected from the group consisting of adalimumab, adalimumab-atto, adalimumab-adaz, adalimumab-afzb, and adalimumab-bwwd.

In some embodiments, the composition further comprises one or more additional agents.

In some embodiments, the one or more additional agents are selected from the group consisting of a nucleic acid, a small molecule, and a polypeptide.

In some embodiments, the one or more additional agents are selected from the group consisting of a corticosteroid, an aminosalicylates, an immunomodulator, a monoclonal antibody, and a biologic.

In some embodiments, the composition is formulated for delivery to a mucus membrane, across a mucus membrane, or a combination thereof.

In some embodiments, the mucus membrane is a nasal membrane, an oral membrane, a vaginal membrane, or a combination thereof.

In some embodiments, the composition is formulated for administered to a gastrointestinal tract.

In some embodiments, the composition is formulated for subcutaneous administration, intravenous administration, topical administration, intrajejunal administration, oral administration, or a combination thereof. In another embodiment, the composition is formulated for intracolonic administration or administration to the colon.

In some embodiments, the composition is formulated for oral administration.

In some embodiments, the composition is formulated in a form selected from the group consisting of a tablet, a pill, a caplet, a capsule, a spray, an aerosol, a syrup, a liquid, and a combination thereof.

In some embodiments, the composition is encapsulated in a capsule.

In some aspects, provided herein is a method of enhancing delivery of a therapeutic agent into a systemic circulation in a subject in need thereof comprising administering a composition comprising the therapeutic agent and at least one ionic liquid to the subject, wherein the therapeutic agent comprises an antibody or an antibody reagent.

In some embodiments, the at least one ionic liquid comprises choline as a cation component.

In some embodiments, the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the at least one ionic liquid comprises a cationic component and an anionic component in the molar ratio of 1;1, 1:2, 1:3, 1:4, 2:1, 3:1, or 4:1.

In some embodiments, the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

In some embodiments, the composition comprises a first ionic liquid and a second ionic liquid, wherein the first ionic liquid and the second ionic liquid are different.

In some embodiments, the first ionic liquid comprises choline as a cation component, the second ionic liquid comprises choline as a cation component, or a combination thereof.

In some embodiments, the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the first ionic liquid and the second ionic liquid comprise: (i) choline-3-phenylpropanoic acid in the molar ratio of 1:2 and choline-glycolic acid in the molar ratio of 2:1, respectively; (ii) choline-3-phenylpropanoic acid in the molar ratio of 1:1 and choline-glycolic acid in the molar ratio of 2:1, respectively; or (iii) choline-3-phenylpropanoic acid in the molar ratio of 1:1 and choline-glycolic acid in the molar ratio of 1:1, respectively.

In some embodiments, the method as provided herein further comprises glycerol.

In some embodiments, the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

In some embodiments, the composition comprises at least one ionic liquid and glycerol in the ratio of: (i) 10%:90%, wherein the at least one ionic liquid comprises choline-3-phenylpropanoic acid in the molar ratio of 1:1; (ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1; (iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1; (iv) 75%:25%, wherein the at least one ionic liquid comprises choline-tartaric acid in the molar ratio of 2:1 or choline-citric acid in the molar ratio of 3:1; or (v) 90%:10%, wherein the at least one ionic liquid comprises choline-tartaric acid in the molar ratio of 2:1.

In some embodiments, the composition comprises the first ionic liquid and the second ionic liquid, and wherein the composition comprises the first ionic liquid, the second ionic liquid, and glycerol in the ratio of: (i) 10%:45%:45%, wherein the first ionic liquid comprises choline-3-phenylpropanoic acid in the molar ratio of 1:2 and the second ionic liquid comprises choline-glycolic acid in the molar ratio of 2:1; or wherein the first ionic liquid comprises choline-3-phenylpropanoic acid in the molar ratio of 1:1 and the second ionic liquid comprises choline-glycolic acid in the molar ratio of 2:1; or (ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

In some embodiments, the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab-adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

In some embodiments, the antibody or the antibody reagent is infliximab or a biosimilar thereof, adalimumab or a biosimilar thereof, or a combination thereof.

In some embodiments, the infliximab or a biosimilar thereof is selected from the group consisting of infliximab, infliximab-dyyb, infliximab-abda, infliximab-qbtx, and infliximab-axxq.

In some embodiments, the adalimumab or a biosimilar thereof is selected from the group consisting of adalimumab, adalimumab-atto, adalimumab-adaz, adalimumab-afzb, and adalimumab-bwwd.

In some embodiments, composition further comprises one or more additional agents.

In some embodiments, the one or more additional agents are selected from the group consisting of a nucleic acid, a small molecule, and a polypeptide.

In some embodiments, the one or more additional agents are selected from the group consisting of a corticosteroid, an aminosalicylates, an immunomodulator, a monoclonal antibody, and a biologic.

In some embodiments, the composition is formulated for delivery to a mucus membrane, across a mucus membrane, or a combination thereof.

In some embodiments, the mucus membrane is a nasal membrane, an oral membrane, a vaginal membrane, or a combination thereof.

In some embodiments, the composition is formulated for administered to a gastrointestinal tract.

In some embodiments, the composition is formulated for subcutaneous administration, intravenous administration, topical administration, intrajejunal administration, oral administration, or a combination thereof. In another embodiment, the composition is formulated for intracolonic administration or administration to the colon.

In some embodiments, the composition is formulated for oral administration.

In some embodiments, the composition is formulated in a form selected from the group consisting of a tablet, a pill, a caplet, a capsule, a spray, an aerosol, a syrup, a liquid, and a combination thereof.

In some embodiments, the composition is encapsulated in a capsule.

In some embodiments, the subject has a disease or a disorder, wherein the disease or the disorder is an inflammatory disease, an autoimmune disease, or a combination thereof.

In some embodiments, the inflammatory disease, the autoimmune disease, or a combination thereof is characterized by a hyperactive immune system.

In some embodiments, the inflammatory disease, the autoimmune disease, or a combination thereof is selected from the group consisting of rheumatoid arthritis, Crohn's disease, ulcerative colitis, psoriasis, psoriatic arthritis, ankylosing spondylitis, inflammatory bowel disease, and a combination thereof.

In some embodiments, the subject has a disease or a disorder, wherein the disease or the disorder is diabetes.

In some embodiments, the method delivers the composition to a mucus membrane, across a mucus membrane, or a combination thereof in the subject.

In some embodiments, the mucus membrane is a nasal membrane, an oral membrane, a vaginal membrane, or a combination thereof.

In some embodiments, the administering comprises administering to a gastrointestinal tract in the subject.

In some embodiments, the administering comprises subcutaneous administration, intravenous administration, topical administration, intrajejunal administration, oral administration, or a combination thereof. In another embodiment, the composition is formulated for intracolonic administration or administration to the colon.

In some embodiments, the administering comprises oral administration.

In some embodiments, the composition is administered in a single dose.

In some embodiments, the composition is administered in multiple doses.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows stability of infliximab in choline-based ionic liquid formulations by enzyme-linked immunosorbent assay (ELISA).

FIG. 2 shows the accumulation of infliximab in intestinal tissue following intrajejunal and intravenous administration of choline-based ionic liquid formulations and infliximab.

FIG. 3 shows the accumulation of infliximab in intestinal tissue following intrajejunal administration of choline-glycolic infliximab formulation relative to intravenous administration of infliximab.

FIG. 4A-4C show the presence of infliximab in intestinal tissue following intrajejunal administration of choline-glycolic infliximab formulation compared to a naïve animal. In FIG. 4A, Amp HQ IHC staining allows visualization of mAb in intestinal tissue with low background (left panel); images are analyzed by colored region using Indica Labs HALO Software, powered by AI to define tissue regions (middle panel); and signal in images is identified (dark dots) and can be used for further quantification and analysis (right panel). FIG. 4B shows high concentration of infliximab signal in intestinal tissue with ionic liquid formulation. FIG. 4C shows 7.5-fold higher anti-TNF-α mAb (infliximab) signal in tissue above background.

FIG. 5 shows stability of infliximab at 75 mg/mL in choline-glycolic formulation by circular dichroism (CD) after 1 week at room temperature.

FIG. 6 shows stability of infliximab at 75 mg/mL in choline-glycolic formulation by differential scanning fluorimetry (DSF) after 1 week at room temperature.

### DETAILED DESCRIPTION OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The term “ionic liquids” as used herein refers to organic salts or mixtures of organic salts which exist in a liquid state. Ionic liquids have been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group); examples include but are not limited to alcohols, fatty acids, and amines. The anionic and the cationic component may be present in any molar ratio.

In some embodiments, the ionic liquid comprises a cationic component and an anionic component in the molar ratio of from about 4:1 to about 1:4. In some embodiments, the ionic liquid comprises a cationic component and an anionic component in the molar ratio of about 4.4:1. In some embodiments, the ionic liquid comprises a cationic component and an anionic component in the molar ratio of about 4.3:1. In some embodiments, the ionic liquid comprises a cationic component and an anionic component in the molar ratio of about 4.2:1. In some embodiments, the ionic liquid comprises a cationic component and an anionic component in the molar ratio of about 4.1:1. In some embodiments, the ionic liquid comprises a cationic component and an anionic component in the molar ratio of about 4.0:1. In some embodiments, the ionic liquid comprises a cationic component and an anionic component in the molar ratio of about 3.9:1. In some embodiments, the ionic liquid comprises a cationic component and an anionic component in the molar ratio of about 3.8:1. In some embodiments, the ionic liquid comprises a cationic component and an anionic component in the molar ratio of about 3.7:1. In some embodiments, the ionic liquid comprises a cationic component and an anionic component in the molar ratio of about 3.6:1. In some embodiments, the ionic liquid comprises a cationic component and an anionic component in the molar ratio of about 3.5:1. In some embodiments, the ionic liquid comprises a cationic component and an anionic component in the molar ratio of about 3.4:1. In some embodiments, the ionic liquid comprises a cationic component and an anionic component in the molar ratio of about 3.3:1. In some embodiments, the ionic liquid comprises a cationic component and an anionic component in the molar ratio of about 3.2:1. In some embodiments, the ionic liquid comprises a cationic component and an anionic component in the molar ratio of about 3.1:1. In some embodiments, the ionic liquid comprises a cationic component and an anionic component in the molar ratio of about 3.0:1. In some embodiments, the ionic liquid comprises a cationic component and an anionic component in the molar ratio of about 2.9:1. In some embodiments, the ionic liquid comprises a cationic component and an anionic component in the molar ratio of about 2.8:1. In some embodiments, the ionic liquid comprises a cationic component and an anionic component in the molar ratio of about 2.7:1. In some embodiments, the ionic liquid comprises a cationic component and an anionic component in the molar ratio of about 2.6:1. In some embodiments, the ionic liquid comprises a cationic component and an anionic component in the molar ratio of about 2.5:1. In some embodiments, the ionic liquid comprises a cationic component and an anionic component in the molar ratio of about 2.4:1. In some embodiments, the ionic liquid comprises a cationic component and an anionic component in the molar ratio of about 2.3:1. In some embodiments, the ionic liquid comprises a cationic component and an anionic component in the molar ratio of about 2.2:1. In some embodiments, the ionic liquid comprises a cationic component and an anionic component in the molar ratio of about 2.1:1. In some embodiments, the ionic liquid comprises a cationic component and an anionic component in the molar ratio of about 2:1. In some embodiments, the ionic liquid comprises a cationic component and an anionic component in the molar ratio of about 1.9:1. In some embodiments, the ionic liquid comprises a cationic component and an anionic component in the molar ratio of about 1.8:1. In some embodiments, the ionic liquid comprises a cationic component and an anionic component in the molar ratio of about 1.7:1. In some embodiments, the ionic liquid comprises a cationic component and an anionic component in the molar ratio of about 1.6:1. In some embodiments, the ionic liquid comprises a cationic component and an anionic component in the molar ratio of about 1.5:1. In some embodiments, the ionic liquid comprises a cationic component and an anionic component in the molar ratio of about 1.4:1. In some embodiments, the ionic liquid comprises a cationic component and an anionic component in the molar ratio of about 1.3:1. In some embodiments, the ionic liquid comprises a cationic component and an anionic component in the molar ratio of about 1.2:1. In some embodiments, the ionic liquid comprises a cationic component and an anionic component in the molar ratio of about 1.1:1. In some embodiments, the ionic liquid comprises a cationic component and an anionic component in the molar ratio of about 1:1. In some embodiments, the ionic liquid comprises a cationic component and an anionic component in the molar ratio of about 1:1.1. In some embodiments, the ionic liquid comprises a cationic component and an anionic component in the molar ratio of about 1:1.2. In some embodiments, the ionic liquid comprises a cationic component and an anionic component in the molar ratio of about 1:1.3. In some embodiments, the ionic liquid comprises a cationic component and an anionic component in the molar ratio of about 1:1.4. In some embodiments, the ionic liquid comprises a cationic component and an anionic component in the molar ratio of about 1:1.5. In some embodiments, the ionic liquid comprises a cationic component and an anionic component in the molar ratio of about 1:1.6. In some embodiments, the ionic liquid comprises a cationic component and an anionic component in the molar ratio of about 1:1.7. In some embodiments, the ionic liquid comprises a cationic component and an anionic component in the molar ratio of about 1:1.8. In some embodiments, the ionic liquid comprises a cationic component and an anionic component in the molar ratio of about 1:1.9. In some embodiments, the ionic liquid comprises a cationic component and an anionic component in the molar ratio of about 1:2. In some embodiments, the ionic liquid comprises a cationic component and an anionic component in the molar ratio of about 1:2.1. In some embodiments, the ionic liquid comprises a cationic component and an anionic component in the molar ratio of about 1:2.2. In some embodiments, the ionic liquid comprises a cationic component and an anionic component in the molar ratio of about 1:2.3. In some embodiments, the ionic liquid comprises a cationic component and an anionic component in the molar ratio of about 1:2.4. In some embodiments, the ionic liquid comprises a cationic component and an anionic component in the molar ratio of about 1:2.5. In some embodiments, the ionic liquid comprises a cationic component and an anionic component in the molar ratio of about 1:2.6. In some embodiments, the ionic liquid comprises a cationic component and an anionic component in the molar ratio of about 1:2.7. In some embodiments, the ionic liquid comprises a cationic component and an anionic component in the molar ratio of about 1:2.8. In some embodiments, the ionic liquid comprises a cationic component and an anionic component in the molar ratio of about 1:2.9. In some embodiments, the ionic liquid comprises a cationic component and an anionic component in the molar ratio of about 1:3.0. In some embodiments, the ionic liquid comprises a cationic component and an anionic component in the molar ratio of about 1:3.1. In some embodiments, the ionic liquid comprises a cationic component and an anionic component in the molar ratio of about 1:3.2. In some embodiments, the ionic liquid comprises a cationic component and an anionic component in the molar ratio of about 1:3.3. In some embodiments, the ionic liquid comprises a cationic component and an anionic component in the molar ratio of about 1:3.4. In some embodiments, the ionic liquid comprises a cationic component and an anionic component in the molar ratio of about 1:3.5. In some embodiments, the ionic liquid comprises a cationic component and an anionic component in the molar ratio of about 1:3.6. In some embodiments, the ionic liquid comprises a cationic component and an anionic component in the molar ratio of about 1:3.7. In some embodiments, the ionic liquid comprises a cationic component and an anionic component in the molar ratio of about 1:3.8. In some embodiments, the ionic liquid comprises a cationic component and an anionic component in the molar ratio of about 1:3.9. In some embodiments, the ionic liquid comprises a cationic component and an anionic component in the molar ratio of about 1:4.0. In some embodiments, the ionic liquid comprises a cationic component and an anionic component in the molar ratio of about 1:4.1. In some embodiments, the ionic liquid comprises a cationic component and an anionic component in the molar ratio of about 1:4.2. In some embodiments, the ionic liquid comprises a cationic component and an anionic component in the molar ratio of about 1:4.3. In some embodiments, the ionic liquid comprises a cationic component and an anionic component in the molar ratio of about 1:4.4. Exemplary molar ratios (cation:anion) include but are not limited to 1:1, 1:2, 1:3, 1:4, 4:1, 3:1, 2:1, 1:1, and ranges between these ratios. In some embodiments, the ionic liquid or solvent exists as a liquid below 100° C. In some embodiments, the ionic liquid or solvent exists as a liquid at room temperature.

In some embodiments, the ionic liquid comprises choline-malonic acid in the molar ratio of 1:1, 1:2, 1:3, 1:4, 4:1, 3:1, 2:1, or 1:1, choline-3-phenylpropanoic acid in the molar ratio of 1:1, 1:2, 1:3, 1:4, 4:1, 3:1, 2:1, or 1:1, choline-3-phenylpropanoic acid in the molar ratio of 1:1, 1:2, 1:3, 1:4, 4:1, 3:1, 2:1, or 1:1, choline-mandelic acid in the molar ratio of 1:1, 1:2, 1:3, 1:4, 4:1, 3:1, 2:1, or 1:1, choline-dl-2-phenylpropionic acid in the molar ratio of 1:1, 1:2, 1:3, 1:4, 4:1, 3:1, 2:1, or 1:1, choline-glycolic acid in the molar ratio of 1:1, 1:2, 1:3, 1:4, 4:1, 3:1, 2:1, or 1:1, choline-malic acid in the molar ratio of 1:1, 1:2, 1:3, 1:4, 4:1, 3:1, 2:1, or 1:1, choline-tartaric acid in the molar ratio of 1:1, 1:2, 1:3, 1:4, 4:1, 3:1, 2:1, or 1:1, choline-3-(4-hydroxyphenyl)propionic acid in the molar ratio of 1:1, 1:2, 1:3, 1:4, 4:1, 3:1, 2:1, or 1:1, choline-decanoic acid in the molar ratio of 1:1, 1:2, 1:3, 1:4, 4:1, 3:1, 2:1, or 1:1, choline-lactic acid in the molar ratio of 1:1, 1:2, 1:3, 1:4, 4:1, 3:1, 2:1, or 1:1, choline-cinnamic acid in the molar ratio of 1:1, 1:2, 1:3, 1:4, 4:1, 3:1, 2:1, or 1:1, choline-acetic acid in the molar ratio of 1:1, 1:2, 1:3, 1:4, 4:1, 3:1, 2:1, or 1:1, choline-citric acid in the molar ratio of 1:1, 1:2, 1:3, 1:4, 4:1, 3:1, 2:1, or 1:1, choline-phosphoric acid in the molar ratio of 1:1, 1:2, 1:3, 1:4, 4:1, 3:1, 2:1, or 1:1, choline-2-octenoic acid in the molar ratio of 1:1, 1:2, 1:3, 1:4, 4:1, 3:1, 2:1, or 1:1, choline-citronellic acid in the molar ratio of 1:1, 1:2, 1:3, 1:4, 4:1, 3:1, 2:1, or 1:1, choline-succinic acid in the molar ratio of 1:1, 1:2, 1:3, 1:4, 4:1, 3:1, 2:1, or 1:1, choline-salicylic acid in the molar ratio of 1:1, 1:2, 1:3, 1:4, 4:1, 3:1, 2:1, or 1:1, or choline-benzoic acid in the molar ratio of 1:1, 1:2, 1:3, 1:4, 4:1, 3:1, 2:1, or 1:1.

The phrase “pharmaceutically acceptable excipient” or “pharmaceutically acceptable carrier” as used herein means a pharmaceutically acceptable material, composition or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, glycerol, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Coadministration includes simultaneous administration in separate compositions, administration at different times in separate compositions, or administration in a composition in which both agents are present.

As used herein, a “drug” or “therapeutic agent,” as interchangeably used herein, refers to any agent which will exert an effect on a target cell or organism. A drug can be selected from a group comprising: chemicals; small organic or inorganic molecules; peptide; protein; or nucleic acid. Non-limiting examples of active compounds contemplated for use in the methods described herein include, but are not limited to, small molecules, polypeptides, nucleic acids, antibodies, vaccines,

In some embodiments of any of the aspects, the active compound can be a therapeutic compound or drug, e.g., an agent or compound which is therapeutically effective for the treatment of at least one condition in a subject. Therapeutic compounds are known in the art for a variety of conditions, see, e.g., the database available on the world wide web at drugs.com or the catalog of FDA-approved compounds available on the world wide web at catalog.data.gov/dataset/drugsfda-database; each of which is incorporated by reference herein in its entirety.

By way of non-limiting example, exemplary antibodies and/or antibody reagents suitable for use as active compounds/therapeutic compounds herein include: abciximab; adalimumab; adlimumab-atto; ado-trastuzumab; ado-trastuzumab emtansine; alemtuzumab; alirocumab; atezolizumab; avelumab; basiliximab; belimumab; bevacizumab; bezlotoxumab; blinatumomab; brentuximab; brentuximab vedotin; brodalumab; canakinumab; capromab; capromab pendetide; certolizumab; certolizumab pegol; cetuximab; daclizumab; daratumumab; denosumab; dinutuximab; dupilumab; durvalumab; eculizumab; elotuzumab; evolocumab; etanercept; etanercept-szzs; golimumab; ibritumomab; ibritumomab tiuxetan; idarucizumab; infliximab; infliximab-abda; infliximab-dyyb; ipilimumab; ixekizumab; mepolizumab; natalizumab; necitumumab; nivolumab; obiltoxaximab; obinutuzumab; ocrelizumab; ofatumumab; olaratumab; omalizumab; palivizumab; panitumumab; pembrolizumab; pertuzumab; ramucriumab; ranibizumab; raxibacumab; reslizumab; rituximab; secukinumab; siltuximab; tocilizumab; trastuzumab; ustekinumab; vedolizumab; sarilumab; guselkumab; inotuzumab ozogamicin; inotuzumab; adalimumab-adbm, gemtuzumab ozogamicin; gemtuzumab; bevacizumab-awwb; benralizumab; emicizumab; emicizumab-kxwh; trastuzumab-dkst; infliximab-qbtx; ibalizumab; ibalizumab-uiyk; tildrakizumab; tildrakizumab-asmn; burosumab; burosumab-twza; erenumab; erenumab-aooe; tositumomab; mogamulizumab; moxetumomab; moxetumomab pasudotox; cemiplimab; polatuzumab; catumaxomab; polatuzumab vedotin; and combinations thereof, including bispecific antibodies made by combining portions of the foregoing.

In some embodiments of any of the aspects, the active compound is a polypeptide. In some embodiments of any of the aspects, the active compound is an antibody or antibody reagent. As used herein, the term “antibody” or “antibody reagent,” as interchangeably used herein, refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. In some embodiments, an antibody comprises multiple chains or a single chain. In some embodiments, an antibody comprises an intact immunoglobulins. In some embodiments, an antibody is naturally driven. In some embodiments, an antibody is recombinantly driven. In some embodiments, an antibody is in the form of a single domain antibody, a maxibody, a minibody, a nanobody, an intrabody, a diabody, a triabody, a tetrabody, and a multispecific antibody.

The term “antibody reagent” or “antibody fragment,” as interchangeably used herein, refers to at least a portion of an intact antibody or recombinant variants thereof. In some embodiments, the antibody reagent or antibody fragment is an antigen binding domain that recognizes and specifically binds to an antigen. In some embodiments, the antibody reagent or antibody fragment encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

As used herein, “inflammatory and autoimmune disease” refers to a disease characterized by an hyperactive immune system. As used throughout, “inflammatory and autoimmune disease ” includes all types including Inflammatory Bowel Disease, Ulcerative Colitis and Crohn's disease unless otherwise specified herein. Inflammatory and autoimmune disease patients are treated by a variety of drugs including oral and topical corticosteroids, aminosalicylates, immunomodulators, monoclonal antibodies and biologics.

### Compositions Comprising Ionic Liquids

In one aspect of any of the embodiments, described herein is a composition comprising at least one ionic liquid comprising 1) an anion which is a carboxylic acid as described herein; and 2) a cation comprising a quaternary ammonium, and a therapeutic agent. In some embodiments, the therapeutic agent comprises an antibody or an antibody reagent. In some embodiments, the composition comprises the antibody or the antibody reagent at the final concentration of at least about 0.0001, about 0.0005, about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 95, about 100, about 150, about 200, about 250, about 300, about 350, about 400, about 450, about 500, about 550, about 600, about 650, about 700, about 750, about 800, about 850, about 900, about 950, about 1000 mg/mL of the antibody or the antibody reagent. In some embodiments, the composition further comprises glycerol. In some embodiments, the composition comprises at least one ionic liquid and glycerol in the ratio of about 100%: about 0%, about 95%: about 5%, about 90%: about 10%, about 85%: about 15%, about 80%: about 20%, about 75%: about 25%, about 70%: about 30%, about 65%: about 35%, about 60%: about 40%, about 55%: about 45%, about 50%: about 50%, about 45%: about 55%, about 40%: about 60%, about 35%: about 65%, about 30%: about 70%, about 25%: about 75%, about 20%: about 80%, about 15%: about 85%, about 10%: about 90%, about 5%: about 95%, about or 0%: about 100%. In some embodiments, the composition comprises at least one ionic liquid and glycerol in the ratio of 100%:0%, 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, 5%:95%, or 0%:100%.

In some embodiments, the composition comprises at least one ionic liquid selected from the ionic liquids listed in Table 1.

In some embodiments, the composition comprises a first ionic liquid and a second ionic liquid. In some embodiments, the first ionic liquid and the second ionic liquid are different.

In some embodiments, the first ionic liquid comprises choline as a cation component. In some embodiments, the second ionic liquid comprises choline as a cation component. In some embodiments, the first ionic liquid comprises choline as a cation component, and the second ionic liquid comprises choline as a cation component. In some embodiments, the first ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid. In some embodiments, the second ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid. In some embodiments, the first ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid, and the second ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

In some embodiments, the first ionic liquid comprises choline-malonic acid in the molar ratio of 1:1, 1:2, 1:3, 1:4, 4:1, 3:1, 2:1, or 1:1, choline-3-phenylpropanoic acid in the molar ratio of 1:1, 1:2, 1:3, 1:4, 4:1, 3:1, 2:1, or 1:1, choline-mandelic acid in the molar ratio of 1:1, 1:2, 1:3, 1:4, 4:1, 3:1, 2:1, or 1:1, choline-dl-2-phenylpropionic acid in the molar ratio of 1:1, 1:2, 1:3, 1:4, 4:1, 3:1, 2:1, or 1:1, choline-glycolic acid in the molar ratio of 1:1, 1:2, 1:3, 1:4, 4:1, 3:1, 2:1, or 1:1, choline-malic acid in the molar ratio of 1:1, 1:2, 1:3, 1:4, 4:1, 3:1, 2:1, or 1:1, choline-tartaric acid in the molar ratio of 1:1, 1:2, 1:3, 1:4, 4:1, 3:1, 2:1, or 1:1, choline-3-(4-hydroxyphenyl)propionic acid in the molar ratio of 1:1, 1:2, 1:3, 1:4, 4:1, 3:1, 2:1, or 1:1, choline-decanoic acid in the molar ratio of 1:1, 1:2, 1:3, 1:4, 4:1, 3:1, 2:1, or 1:1, choline-lactic acid in the molar ratio of 1:1, 1:2, 1:3, 1:4, 4:1, 3:1, 2:1, or 1:1, choline-cinnamic acid in the molar ratio of 1:1, 1:2, 1:3, 1:4, 4:1, 3:1, 2:1, or 1:1, choline-acetic acid in the molar ratio of 1:1, 1:2, 1:3, 1:4, 4:1, 3:1, 2:1, or 1:1, choline-citric acid in the molar ratio of 1:1, 1:2, 1:3, 1:4, 4:1, 3:1, 2:1, or 1:1, choline-phosphoric acid in the molar ratio of 1:1, 1:2, 1:3, 1:4, 4:1, 3:1, 2:1, or 1:1, choline-2-octenoic acid in the molar ratio of 1:1, 1:2, 1:3, 1:4, 4:1, 3:1, 2:1, or 1:1, choline-citronellic acid in the molar ratio of 1:1, 1:2, 1:3, 1:4, 4:1, 3:1, 2:1, or 1:1, choline-succinic acid in the molar ratio of 1:1, 1:2, 1:3, 1:4, 4:1, 3:1, 2:1, or 1:1, choline-salicylic acid in the molar ratio of 1:1, 1:2, 1:3, 1:4, 4:1, 3:1, 2:1, or 1:1, or choline-benzoic acid in the molar ratio of 1:1, 1:2, 1:3, 1:4, 4:1, 3:1, 2:1, or 1:1. In some embodiments, the second ionic liquid comprises choline-malonic acid in the molar ratio of 1:1, 1:2, 1:3, 1:4, 4:1, 3:1, 2:1, or 1:1, choline-3-phenylpropanoic acid in the molar ratio of 1:1, 1:2, 1:3, 1:4, 4:1, 3:1, 2:1, or 1:1, choline-mandelic acid in the molar ratio of 1:1, 1:2, 1:3, 1:4, 4:1, 3:1, 2:1, or 1:1, choline-dl-2-phenylpropionic acid in the molar ratio of 1:1, 1:2, 1:3, 1:4, 4:1, 3:1, 2:1, or 1:1, choline-glycolic acid in the molar ratio of 1:1, 1:2, 1:3, 1:4, 4:1, 3:1, 2:1, or 1:1, choline-malic acid in the molar ratio of 1:1, 1:2, 1:3, 1:4, 4:1, 3:1, 2:1, or 1:1, choline-tartaric acid in the molar ratio of 1:1, 1:2, 1:3, 1:4, 4:1, 3:1, 2:1, or 1:1, choline-3-(4-hydroxyphenyl)propionic acid in the molar ratio of 1:1, 1:2, 1:3, 1:4, 4:1, 3:1, 2:1, or 1:1, choline-decanoic acid in the molar ratio of 1:1, 1:2, 1:3, 1:4, 4:1, 3:1, 2:1, or 1:1, choline-lactic acid in the molar ratio of 1:1, 1:2, 1:3, 1:4, 4:1, 3:1, 2:1, or 1:1, choline-cinnamic acid in the molar ratio of 1:1, 1:2, 1:3, 1:4, 4:1, 3:1, 2:1, or 1:1, choline-acetic acid in the molar ratio of 1:1, 1:2, 1:3, 1:4, 4:1, 3:1, 2:1, or 1:1, choline-citric acid in the molar ratio of 1:1, 1:2, 1:3, 1:4, 4:1, 3:1, 2:1, or 1:1, choline-phosphoric acid in the molar ratio of 1:1, 1:2, 1:3, 1:4, 4:1, 3:1, 2:1, or 1:1, choline-2-octenoic acid in the molar ratio of 1:1, 1:2, 1:3, 1:4, 4:1, 3:1, 2:1, or 1:1, choline-citronellic acid in the molar ratio of 1:1, 1:2, 1:3, 1:4, 4:1, 3:1, 2:1, or 1:1, choline-succinic acid in the molar ratio of 1:1, 1:2, 1:3, 1:4, 4:1, 3:1, 2:1, or 1:1, choline-salicylic acid in the molar ratio of 1:1, 1:2, 1:3, 1:4, 4:1, 3:1, 2:1, or 1:1, or choline-benzoic acid in the molar ratio of 1:1, 1:2, 1:3, 1:4, 4:1, 3:1, 2:1, or 1:1.

In some embodiments, the composition further comprises glycerol. In some embodiments, the composition comprises the first ionic liquid, the second ionic liquid, and glycerol in the ratio of 100%:0%:0%, 95%:5%:0%, 90%:10%:0%, 85%:15%:0%, 80%:20%:0%, 75%:25%:0%, 70%:30:0%, 65%:35%:0%, 60%:40%:0%, 55%:45%:0%, 50%:50%:0%, 45%:55%:0%, 40%:60%:0%, 35%:65%:0%, 30%:70%:0%, 25%:75%:0%, 20%:80%:0%, 15%:85%:0%, 10%:90%:0%, 5%:95%:0%, 0%:100%:0%; 0%:95%:5%, 5%:90%:5%, 10%:85%:5%, 15%:80%:5%, 20%:75%:5%, 25%:70%:5%, 30%:65%:5%, 35%:60%:5%, 40%:55%:5%, 45%:50%:5%, 50%:45%:5%, 55%:40%:5%, 60%:35%:5%, 65%:30%:5%, 70%:25%:5%, 75%:20%:5%, 80%:15%:5%, 85%:10%:5%, 90%:5%:5%, 95%:0%:5%; 0%:90%:10%, 5%:85%:10%, 10%:80%:10%, 15%:75%:10%, 20%:70%:10%, 25%:65%:10%, 30%:60%:10%, 35%:55%:10%, 40%:50%:10%, 45%:45%:10%, 50%:40%:10%, 55%:35%:10%, 60%:30%:10%, 65%:25%:10%, 70%:20%:10%, 75%:15%:10%, 80%:10%:10%, 85%:5%:10%, 90%:0%:10%; 0%:85%:15%, 5%:80%:15%, 10%:75%:15%, 15%:70%:15%, 20%:65%:15%, 25%:60%:15%, 30%:55%:15%, 35%:50%:15%, 40%:45%:15%, 45%:40%:15%, 50%:35%:15%, 55%:30%:15%, 60%:25%:15%, 65%:20%:15%, 70%:15%:15%, 75%:10%:15%, 80%:5%:15%, 85%:0%:15%; 0%:80%:20%, 5%:75%:20%, 10%:70%:20%, 15%:65%:20%, 20%:60%:20%, 25%:55%:20%, 30%:50%:20%, 35%:45%:20%, 40%:40%:20%, 45%:35%:20%, 50%:30%:20%, 55%:25%:20%, 60%:20%:20%, 65%:15%:20%, 70%:10%:20%, 75%:5%:20%, 80%:0%:20%; 0%:75%:25%, 5%:70%:25%, 10%:65%:25%, 15%:60%:25%, 20%:55%:25%, 25%:50%:25%, 30%:45%:25%, 35%:40%:25%, 40%:35%:25%, 45%:30%:25%, 50%:25%:25%, 55%:20%:25%, 60%:15%:25%, 65%:10%:25%, 70%:5%:25%, 75%:0%:25%; 0%:70%:30%, 5%:65%:30%, 10%:60%:30%, 15%:55%:30%, 20%:50%:30%, 25%:45%:30%, 30%:40%:30%, 35%:35%:30%, 40%:30%:30%, 45%:25%:30%, 50%:20%:30%, 55%:15%:30%, 60%:10%:30%, 65%:5%:30%, 70%:0%:30%; 0%:65%:35%, 5%:60%:35%, 10%:55%:35%, 15%:50%:35%, 20%:45%:35%, 25%:40%:35%, 30%:35%:35%, 35%:30%:35%, 40%:25%:35%, 45%:20%:35%, 50%:15%:35%, 55%:10%:35%, 60%:5%:35%, 65%:0%:35%; 0%:60%:40%, 5%:55%:40%, 10%:50%:40%, 15%:45%:40%, 20%:40%:40%, 25%:35%:40%, 30%:30%:40%, 35%:25%:40%, 40%:20%:40%, 45%:15%:40%, 50%:10%:40%, 55%:5%:40%, 60%:0%:40%; 0%:55%:45%, 5%:50%:45%, 10%:45%:45%, 15%:40%:45%, 20%:35%:45%, 25%:30%:45%, 30%:25%:45%, 35%:20%:45%, 40%:15%:45%, 45%:10%:45%, 50%:5%:45%, 55%:0%:45%; 0%:50%:50%, 5%:45%:50%, 10%:40%:50%, 15%:35%:50%, 20%:30%:50%, 25%:25%:50%, 30%:20%:50%, 35%:15%:50%, 40%:10%:50%, 45%:5%:50%, 50%:0%:50%; 0%:45%:55%, 5%:40%:55%, 10%:35%:55%, 15%:30%:55%, 20%:25%:55%, 25%:20%:55%, 30%:15%:55%, 35%:10%:55%, 40%:5%:55%, 45%:0%:55%; 0%:40%:60%, 5%:35%:60%, 10%:30%:60%, 15%:25%:60%, 20%:20%:60%, 25%:15%:60%, 30%:10%:60%, 35%:5%:60%, 40%:0%:60%; 0%:35%:65%, 5%:30%:65%, 10%:25%:65%, 15%:20%:65%, 20%:15%:65%, 25%:10%:65%, 30%:5%:65%, 35%:0%:65%; 0%:30%:70%, 5%:25%:70%, 10%:20%:70%, 15%:15%:70%, 20%:10%:70%, 25%:5%:70%, 30%:0%:70%; 0%:25%:75%, 5%:20%:75%, 10%:15%:75%, 15%:10%:75%, 20%:5%:75%, 25%:0%:75%; 0%:20%:80%, 5%:15%:80%, 10%:10%:80%, 15%:5%:80%, 20%:0%:80%; 0%:15%:85%, 5%:10%:85%, 10%:5%:85%, 15%:0%:85%; 0%:10%:90%, 5%:5%:90%, 10%:0%:90%:0%:5%:95%, 5%:0%:95%; or 0%:0%:100%.

Provided herein, in some embodiments, are compositions comprising ionic liquids useful in the treatment of certain diseases and disorders.

### Formulation

In some embodiments, an ionic liquid provided herein is formulated in combination with a one or more drugs. In some embodiments, the ionic liquid can be combined with another solvent to enhance solubility and/or delivery. The solvent may be aqueous or non-aqueous. In some embodiments, the purpose of the solvent is to control the dose of the ionic liquid experienced by the mucus membrane or the gastrointestinal tract. Dilution of the ionic liquid by the solvent can serve the purpose of delivering a safe dose to the subject. In some embodiments, the purpose of the solvent is to improve solubility of the one or more drugs. Such improvements may come from the ability of the solvent to control the physicochemical environment of the ionic liquid to match the chemical properties of the one or more drugs. In some embodiments, the solvent may serve the purpose of improving the delivery across the mucosal membrane.

The solvents used may include without limitation: sterile water, saline solution, glycerin, propylene glycol, ethanol, oils, ethyl oleate, isopropyl myristate, benzyl benzoate, or surfactants.

In some embodiments, the solvent is chosen so as to not adversely impact the compatibility of the ionic liquid with the capsule.

In some embodiments, the one or more drugs may form micelles or other self-assembled structures. In some embodiments, such structures may occur only in the presence of ionic liquids.

In some embodiments, the one or more drugs is a nucleic acid molecule. A nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other natural or synthetic nucleic acid molecule intended for delivery to an organism.

In any of the embodiments, the one or more drugs may be designed with the intent of treating a local tissue, e.g., the mucosal membrane of the intestine, the lining of the intestine, treating a distant tissue, e.g., the liver, or entering systemic circulation.

In some embodiments, a composition as described herein, e.g., a composition comprising ionic liquids and one or more drugs, can further comprise a pharmaceutically acceptable excipient. Suitable excipients include, for example, water, saline, glycerol, ethanol, or the like, and combinations thereof. In addition, if desired, the composition can contain minor amounts of additional excipients such as emulsifying agents, surfactants, pH buffering agents, and the like, which enhance the effectiveness of the one or more drugs.

In some embodiments, the composition comprising an ionic liquid may be further encapsulated in a dosage form designed to facilitate delivery to an organism. Non-limiting examples of such dosage forms include capsules, tablets, and syrups.

In some embodiments, formulation may require excipients sugars (such as lactose), starches (such as corn starch), cellulose, cellulose derivatives (such as sodium carboxymethyl cellulose), gelatin, and other compatible substances.

In some embodiments, a composition comprising an ionic liquid described herein further comprises one or more additional agents. In some embodiments, the one or more additional agents are selected from a nucleic acid, a small molecule, and a polypeptide. In some embodiments, the one or more additional agents comprise a nucleic acid. In some embodiments, the one or more additional agents comprise a small molecule. In some embodiments, the one or more additional agents comprise a polypeptide. In some embodiments the polypeptide comprises an antibody. In some embodiments, the antibody comprises any one selected from Fragment Antigen-binding (Fab, F(ab′)₂), single chain variable fragment (scFv), and nanobodies.

### Ionic Liquids for the Treatment of Diseases and Disorders

The term “ionic liquids (ILs)” as used herein refers to organic salts or mixtures of organic salts which are in liquid state at room temperature. This class of solvents has been shown to be useful in a variety of fields, including in industrial processing, catalysis, pharmaceuticals, and electrochemistry.

In some embodiments, the composition comprises the ionic liquid at a concentration of at least 0.1% weight per volume. In some embodiments, the composition comprises the ionic liquid at a concentration of at least 0.05 M. In some embodiments, the ionic liquid comprises a cationic component and an anionic component in the molar ratio of from about 4:1 to about 1:4.

The ionic liquids contain at least one anionic and at least one cationic component. Ionic liquids can comprise an additional hydrogen bond donor (i.e. any molecule that can provide an —OH or an ˜NH group), examples include but are not limited to alcohols, fatty acids, and amines. The at least one anionic and at least one cationic component may be present in any molar ratio. Exemplary molar ratios (cation:anion) include but are not limited to 1:4, 1:2, 2:1, 1:3, 3:1, 2:3, 3:2, 4:1 and ranges between these ratios. For further discussion of ionic liquids, see, e.g., Hough, et ah , “The third evolution of ionic liquids: active pharmaceutical ingredients”, New Journal of Chemistry, 31: 1429 (2007) and Xu, et al., “Ionic Liquids: Ion Mobilities, Glass Temperatures, and Fragilities”, Journal of Physical Chemistry B, 107(25): 6170-6178 (2003); each of which is incorporated by reference herein in its entirety. In some embodiments of any of the aspects, the ionic liquid or solvent exists as a liquid below 100° C. In some embodiments of any of the aspects, the ionic liquid or solvent exists as a liquid at room temperature.

In some embodiments, the composition further comprises one or more additional agents. In some embodiments, the one or more additional agents are selected from a nucleic acid, a small molecule, and a polypeptide. In some embodiments, the one or more additional agents comprise a nucleic acid. In some embodiments, the one or more additional agents comprise a small molecule. In some embodiments, the one or more additional agents comprise a polypeptide. In some embodiments, the one or more additional agents comprise an antibody. In some embodiments, the one or more additional agents comprise a nanobody.

In some embodiments, the one or more additional agents are selected from a antibodies that bind specific cytokines or cell surface receptors. In some embodiments, the one or more additional agents are selected from infliximab, adalimumab, golimumab and their respective biosimilars.

Provided herein, in another aspect, is a pharmaceutical composition comprising a composition described herein and a pharmaceutically acceptable excipient.

Provided herein, in some embodiments, is a method of treating a metabolic disease or disorder in a subject in need thereof, comprising administering a composition comprising an ionic liquid. Metabolic disorders include but are not limited to obesity, diabetes, fatty liver disease, or non-alcoholic fatty liver disease.

Provided herein, in some embodiments, is the use of ionic liquids for treating diabetes by oral administration. Oral administration can be achieved in any one of the dosing forms including pills, caplets, capsules, aerosol sprays, or liquids. The ionic liquid or the one or more drugs to be delivered with the ionic liquid can be encapsulated in a capsule. The ionic liquid with the dosing form may be present in any of the physical forms including a clear neat ionic liquid, a homogenous mixture of an ionic liquid with a pharmaceutically acceptable diluent, an emulsion, or a suspension. The oral dose can also be given as a syrup, a spray, or an aerosol. The composition of any oral dose disclosed herein may contain a predetermined amount of ionic liquid and optionally one or more drugs, and may be prepared by methods of pharmacy well known to those skilled in the art.

In some embodiments, described herein is a method of treatment of inflammatory and autoimmune diseases comprising orally administering an oral formulation of infliximab in combination with an ionic liquid.

As described herein, ionic liquids are able to safely carry active compounds across the mucus membranes encountered during oral administration.

As described in the examples herein, when administered together with one or more drugs, ionic liquids solubilize the one or more drugs and result in enhanced delivery into systemic circulation. Accordingly, they are particularly suitable as delivery vehicles to and/or across mucus membranes.

In some embodiments, provided herein is a method of delivery of one or more drugs, the method comprising administering the one or more drugs in combination with an ionic liquid to a mucus membrane, e.g., a nasal, oral, or vaginal membrane.

Provided herein, in one aspect, is a method of treating a disease or disorder in a subject in need thereof, comprising administering to the subject a therapeutically effective amount of a composition comprising an ionic liquid.

In some embodiments, the disease or disorder is inflammatory and autoimmune diseases . In some embodiments, the disease or disorder is inflammatory bowel disease (IBD). In some embodiments, the disease or disorder is ulcerative colitis. In some embodiments, the disease or disorder is Crohn's disease.

Provided herein, in another aspect, is a method of treating inflammatory and autoimmune disease, comprising administering to the subject a therapeutically effective amount of a composition comprising an ionic liquid.

In some embodiments, the composition is administered via subcutaneous, intravenous, or oral administration. In some embodiments, the composition is administered via oral administration. In some embodiments, the composition is administered as a liquid-filled capsule. In some embodiments, the composition is administered in a single dose. In some embodiments, the composition is administered in multiple doses. In some embodiments, the composition is administered to a mucus membrane.

### Anti-TNF-α Monoclonal Antibodies

Infliximab and Adalimumabs are monoclonal antibodies known to bind TNF-α. As TNF-α plays an important role in inflammation, antibodies that block the action of TNF-α have been used to treat inflammatory and autoimmune disease. Examples of diseases for which anti-TNF-α is used include Rheumatoid Arthritis, Crohn's Disease, Ulcerative Colitis, Psoriasis, Psoriatic Arthritis and Ankylosing Spondylitis.

Many biosimilars that demonstrate similar efficacy to the original reference products (Infliximab, Adalimumab) have also been developed and are also used to treat inflammatory and autoimmune disease. Infliximab biosimilars include Inflectra (Infliximab-dyyb), Renflexis (Infliximab-abda), Ixifi (Infliximab-qbtx), Avsola (infliximab-axxq). Adalimumab biosimilars include Amjevita (Adalimumab-atto), Hyrimoz (adalimumab-adaz), Abrilada (adalimumab-afzb), Hadlima (adalimumab-bwwd).

In some embodiments, described herein is a method of treatment of diabetes comprising orally administering an oral formulation of Infliximab or biosimilar thereof in combination with ionic liquid.

In some embodiments, described herein is a method of treatment of diabetes comprising orally administering an oral formulation of Adalimumab or biosimilar thereof in combination with ionic liquid.

### EXAMPLES

### General

All animal experiments were performed in accordance with the animal care committee guidelines and the Guide for the Care and Use of Animals of the Institute of Laboratory Animal Resources, National Research Council.

### Example 1: Preparation of Ionic Liquids

Several ionic liquids comprising choline as a cation and various anions were synthesized. To prepare ionic liquids, 4, 3, 2, 1, 0.5, or 0.33 equivalents of choline bicarbonate (80 wt % solution) were added to neat carboxylic acid anion in a 250-mL round bottom flask. For anions not miscible with the aqueous choline bicarbonate solution, a co-solvent, such as ethanol, was added until a homogenous mixture formed. The mixture was stirred at room temperature until CO₂ evolution ceased. Solvent was removed by rotary evaporation at 60° C. for 20 minutes, and each product was dried in a vacuum oven for 48 hours at 60° C.

### Example 2: Short-term Stability of Infliximab in Ionic Liquid Formulations by ELISA

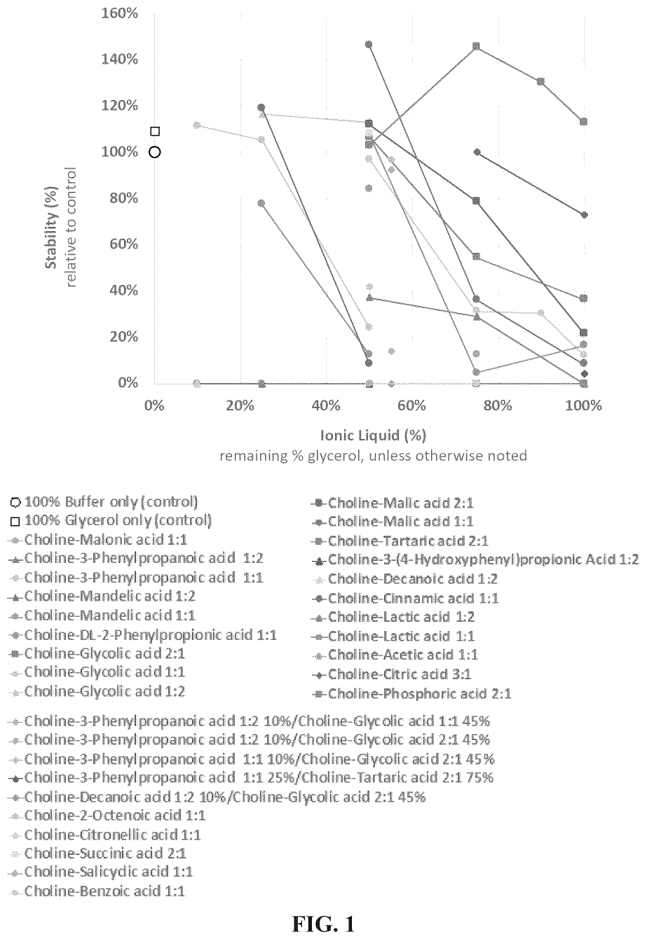

Ionic liquid formulations were prepared using some percentage of ionic liquid and some percentage of glycerol with a final concentration of 0.1 mg/mL of antibody (infliximab, a chimeric monoclonal anti-TNF-α antibody purchased from Novus Biologicals). Formulation compositions are described in Table 1. Antibody-containing formulations remained at room temperature for at least one hour after mixing. Formulations were dialyzed using 10 mM sodium phosphate buffer pH 7.4 and 10 kDa membrane molecular-weight cutoff Thermo Scientific™ Slide-A-Lyzer™ G2 dialysis cassettes for 48 h. Stability of the dialyzed samples were assessed using an Infliximab enzyme-linked immunoassay (ELISA) (purchased from Abcam). Binding and ultimately signal from this assay will occur only if the antibody (infliximab) remains intact and can bind to the target. Percent stability was determined for each formulation relative to infliximab in buffer only condition shown in FIG. 1 and Table 1. Bolded rows in Table 1 denote formulations which resulted in infliximab stability >90%. Currently, it is not obvious why certain ionic liquids containing different anions or different ratios of anions or different amounts of glycerol do not impact the stability of the infliximab antibody. With very few exceptions, formulations containing neat (100%) ionic liquid impact the stability of the antibody; however, the stability becomes less impacted by reducing the ionic liquid percentage. Some formulations contain 50-75% ionic liquid and do not impact the antibody's ability to bind to its target using this ELISA. Of 30 formulations tested, 19 formulations (63%) resulted in >90% stability of infliximab.

TABLE 1
Stability assessment of infliximab in various choline-based ionic
liquid formulations

| Formulation | Glycerol % | IL % | Stability % |
|---|---|---|---|
| 100% Buffer only (control) | 0% | 0% | 100% |
| 100% Glycerol only (control) | 100% | 0% | 109% |
| Choline-Malonic acid 1:1 | 0% | 100% | 0% |
| Choline-Malonic acid 1:1 | 50% | 50% | 0% |
| Choline-Malonic acid 1:1 | 75% | 25% | 0% |
| Choline-Malonic acid 1:1 | 90% | 10% | 0% |
| Choline-3-Phenylpropanoic acid 1:2 | 0% | 100% | 0% |
| Choline-3-Phenylpropanoic acid 1:2 | 50% | 50% | 0% |
| Choline-3-Phenylpropanoic acid 1:2 | 75% | 25% | 0% |
| Choline-3-Phenylpropanoic acid 1:2 | 90% | 10% | 0% |
| Choline-3-Phenylpropanoic acid 1:1 | 50% | 50% | 24% |
| Choline-3-Phenylpropanoic acid 1:1 | 75% | 25% | 105% |
| Choline-3-Phenylpropanoic acid 1:1 | 90% | 10% | 112% |
| Choline-Mandelic acid 1:2 | 50% | 50% | 0% |
| Choline-Mandelic acid 1:1 | 50% | 50% | 84% |
| Choline-DL-2-Phenylpropionic | 50% | 50% | 13% |
| acid 1:1 | | | |
| Choline-DL-2-Phenylpropionic | 75% | 25% | 77% |
| acid 1:1 | | | |
| Choline-Glycolic acid 1:1 | 0% | 100% | 12% |
| Choline-Glycolic acid 1:1 | 10% | 90% | 30% |
| Choline-Glycolic acid 1:1 | 25% | 75% | 31% |
| Choline-Glycolic acid 1:1 | 50% | 50% | 97% |
| Choline-Glycolic acid 2:1 | 0% | 100% | 37% |
| Choline-Glycolic acid 2:1 | 25% | 75% | 55% |
| Choline-Glycolic acid 2:1 | 50% | 50% | 107% |
| Choline-Glycolic acid 1:2 | 50% | 50% | 113% |
| Choline-Glycolic acid 1:2 | 75% | 25% | 117% |
| Choline-Malic acid 2:1 | 0% | 100% | 22% |
| Choline-Malic acid 2:1 | 25% | 75% | 79% |
| Choline-Malic acid 2:1 | 50% | 50% | 112% |
| Choline-Malic acid 1:1 | 0% | 100% | 9% |
| Choline-Malic acid 1:1 | 25% | 75% | 36% |
| Choline-Malic acid 1:1 | 50% | 50% | 146% |
| Choline-Tartaric acid 2:1 | 0% | 100% | 113% |
| Choline-Tartaric acid 2:1 | 10% | 90% | 130% |
| Choline-Tartaric acid 2:1 | 25% | 75% | 145% |
| Choline-Tartaric acid 2:1 | 50% | 50% | 103% |
| Choline-3-(4-Hydroxyphenyl) | 50% | 50% | 0% |
| propionic Acid 1:2 | | | |
| Choline-Decanoic acid 1:2 | 90% | 10% | 0% |
| Choline-Decanoic acid 1:2 10%/ | 45% | 55% | 0% |
| Choline-Glycolic acid 2:1 45% | | | |
| Choline-3-Phenylpropanoic acid 1:2 | 45% | 55% | 14% |
| 10%/Choline-Glycolic acid 1:1 45% | | | |
| Choline-3-Phenylpropanoic acid 1:2 | 45% | 55% | 93% |
| 10%/Choline-Glycolic acid 2:1 45% | | | |
| Choline-3-Phenylpropanoic acid 1:1 | 0% | 100% | 4% |
| 25%/Choline-Tartaric acid 2:1 75% | | | |
| Choline-3-Phenylpropanoic acid 1:1 | 45% | 55% | 97% |
| 10%/Choline-Glycolic acid 2:1 45% | | | |
| Choline-3-Phenylpropanoic acid 1:1 | 50% | 50% | 111% |
| 10%/Choline-Glycolic acid 1:1 40% | | | |
| Choline-3-Phenylpropanoic acid 1:1 | 50% | 50% | 48% |
| 25%/Choline-Glycolic acid 1:1 25% | | | |
| Choline-Lactic acid 1:2 | 0% | 100% | 0% |
| Choline-Lactic acid 1:2 | 25% | 75% | 29% |
| Choline-Lactic acid 1:2 | 50% | 50% | 37% |
| Choline-Lactic acid 1:1 | 0% | 100% | 17% |
| Choline-Lactic acid 1:1 | 25% | 75% | 5% |
| Choline-Lactic acid 1:1 | 50% | 50% | 107% |
| Choline-Cinnamic acid 1:1 | 75% | 25% | 119% |
| Choline-Cinnamic acid 1:1 | 50% | 50% | 9% |
| Choline-Acetic acid 1:1 | 25% | 75% | 13% |
| Choline-Citric acid 3:1 | 0% | 100% | 73% |
| Choline-Citric acid 3:1 | 25% | 75% | 100% |
| Choline-Phosphoric acid 2:1 | 0% | 100% | 0% |
| Choline-Phosphoric acid 2:1 | 25% | 75% | 0% |
| Choline-2-Octenoic acid 1:1 | 25% | 75% | 0% |
| Choline-2-Octenoic acid 1:1 | 50% | 50% | 0% |
| Choline-Citronellic acid 1:1 | 25% | 75% | 0% |
| Choline-Citronellic acid 1:1 | 50% | 50% | 0% |
| Choline-Succinic acid 2:1 | 50% | 50% | 108% |
| Choline-Salicylic acid 1:1 | 50% | 50% | 0% |
| Choline-Benzoic acid 1:1 | 50% | 50% | 42% |

### Example 3: Local Delivery of Infliximab with Various Choline-based Ionic Liquids

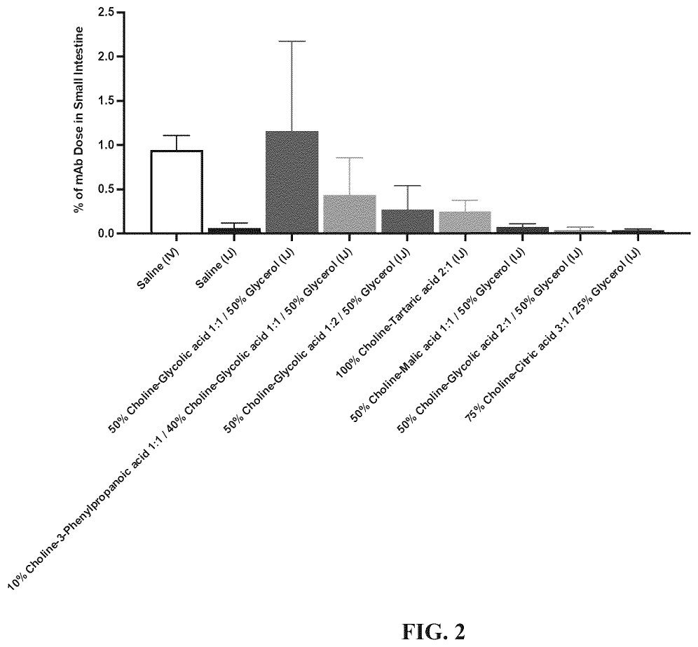

Adult non-diabetic male Wistar rats were fasted overnight but given free access to water and subsequently dosed via intrajejunal injection (IJ) with infliximab and one of various choline-based ionic liquids, dosed via IJ with infliximab and saline, or dosed via intravenous injection (IV) with infliximab and saline. Rats were sacrificed after 5 h at which time small intestines were excised from each animal and washed with PBS. Tissue was homogenized, and infliximab in the local small intestinal tissue was quantified by LC-MS/MS, shown in FIG. 2.

By IV, infliximab in saline had 0.95% of the injected antibody dose in the small intestinal tissue at 5 h. Compared to the negative control of infliximab in saline by IJ, infliximab had a much lower penetration into the local tissue as demonstrated by only 0.06% of the infliximab dose present.

The amount of infliximab delivered was dependent on the composition of the ionic liquid formulation. Formulations containing 75% Choline-Citric acid 3:1/25% Glycerol and 50% Choline-Glycolic acid 2:1/50% Glycerol yielded low local concentrations of infliximab in local tissue: 0.03% and 0.04% of the injected dose, respectively. Modest improvements in local tissue delivery of infliximab were observed with 50% Choline-Malic acid 1:1/50% Glycerol, 100% Choline-Tartaric acid 2:1, 50% Choline-Glycolic acid 1:2/50% Glycerol, and 10% Choline-3-Phenylpropanoic acid 1:1/40% Choline-Glycolic acid 1:1/50% Glycerol were delivery of infliximab in local tissue yielded 0.07%, 0.25%, 0.27%, and 0.44%, respectively. Unexpectedly, a formulation containing 50% Choline-Glycolic 1:1/50% Glycerol with infliximab delivered 1.16% of the injected dose to the local tissue, which is commensurate with dosing IV of infliximab in saline.

### Example 4: Local Delivery of Infliximab with Choline-Glycolic Comparable Delivery to Intravenous Injection Without Corresponding Systemic Exposure

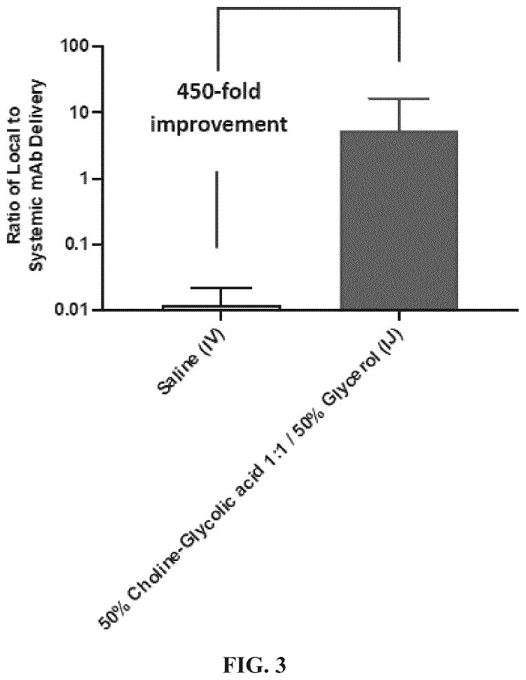

Adult non-diabetic male Wistar rats were fasted overnight but given free access to water and subsequently dosed via intrajejunal injection (IJ) with infliximab and 50% Choline-Glycolic 1:1/50% Glycerol or dosed via intravenous injection (IV) with infliximab and saline. About 250 μL of blood was collected at regular intervals in order to determine the infliximab plasma concentration. Rats were sacrificed after 5 h at which time small intestines were excised from each animal and washed with PBS. Tissue was homogenized, and infliximab in the local small intestinal tissue as well as in plasma was quantified by LC-MS/MS. The concentration of infliximab in local small intestinal tissue was calculated by dividing the total antibody amount in nanograms by the total tissue analyzed in grams. The concentration of infliximab in systemic circulation was determined by calculating the area under the curve (AUC) for each condition having units of nanogram times hour divided by milliliter. The ratio of local to systemic concentration of infliximab was plotted for both conditions, shown in FIG. 3.

By IV, the ratio local to systemic concentration of monoclonal antibody (mAb) infliximab in saline was 0.0117. Surprisingly, despite having commensurate concentration of infliximab in local tissue as outlined in the previous example, a formulation containing 50% Choline-Glycolic 1:1/50% Glycerol with infliximab yielded a ratio of local to systemic concentration of infliximab to be 5.43, a greater than 450-fold improvement in local concentration compared to IV administration. This has important implications for such gastrointestinal tract diseases where current treatments are limited to injection, achieve a low percentage of antibody at the target disease site like the small intestine for such diseases like inflammatory bowel disease, yet suffer from off-target effects due to high systemic exposure of the antibody drug. This ionic liquid formulation can deliver commensurate concentration of infliximab in local tissue while limiting the amount that reaches systemic circulation as compared to conventional injection-based approaches.

### Example 5: Visualization of Local Delivery of Infliximab with Choline-Glycolic

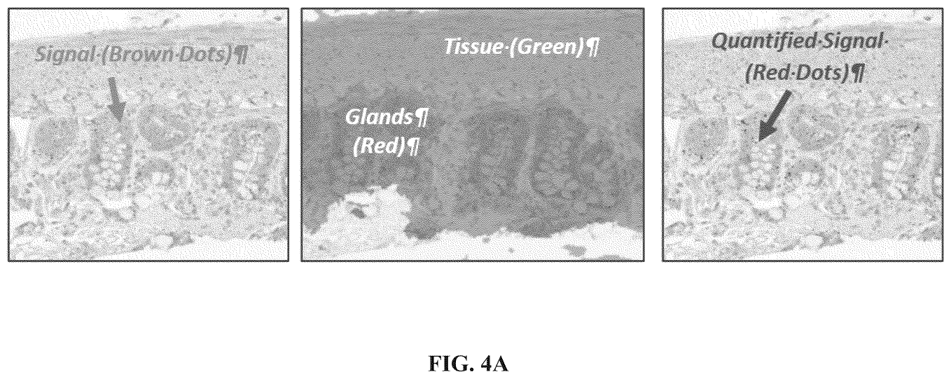

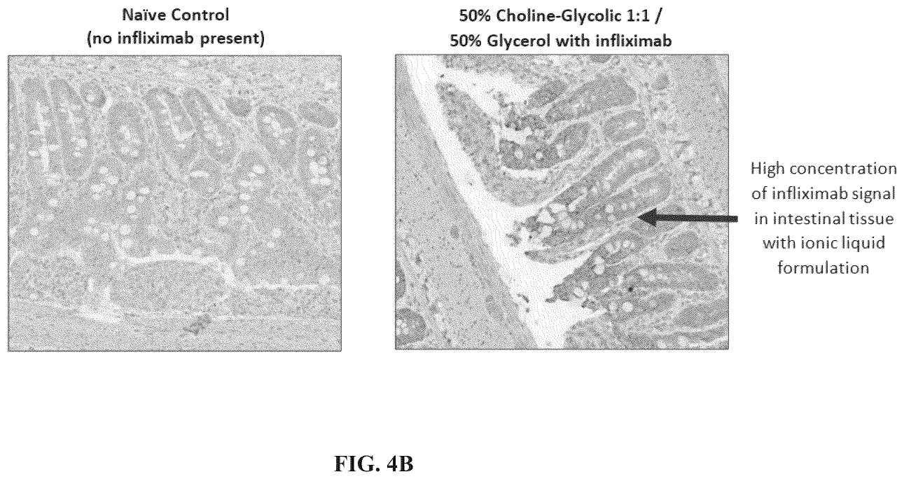

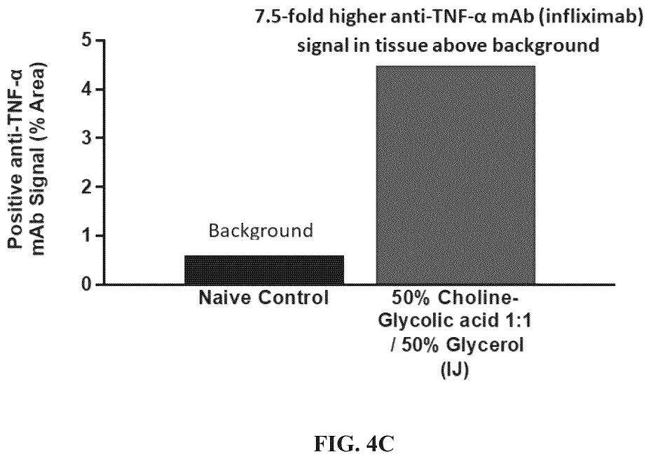

Adult non-diabetic male Wistar rats were fasted overnight but given free access to water and subsequently dosed via intrajejunal injection (IJ) with infliximab and 50% Choline-Glycolic 1:1/50% Glycerol or not dosed (as a naïve control). Rats were sacrificed after 5 h at which time small intestines were excised from each animal and washed with PBS. The swill roll technique was performed on each Intestinal tissue, fixed in 10% neutral buffered formalin, and embedded in paraffin. For detection of infliximab, Amp HQ IHC (Ventana Systems), a tyramide signal amplification detection kit, was used on the tissue samples using a Ventana Discovery Ultra Platform. An anti-human IgG Fc rabbit polyclonal antibody conjugate HPR (1:1000) (Abcam) was used for detection of infliximab has this antibody has a human constant region. Images were analyzed using Indica Labs HALO Software, powered by AI to define regions in the tissue, for each of the conditions tested, as shown in FIG. 4A. This technique allowed for visualization of infliximab in small intestinal tissue with low background, as shown in FIG. 4B. 7.5-fold higher anti-TNF-α monoclonal antibody (infliximab) signal was observed in tissue above background, as shown in FIG. 4C.

### Example 6: Stability of Infliximab at High Concentrations in Choline-Glycolic by Circular Dichroism (CD) After 1 Week at Room Temperature

A formulation containing 50% Choline-Glycolic 1:1 and 50% glycerol with final concentration 75 mg/mL of antibody was prepared (infliximab, a chimeric monoclonal anti-TNF-α antibody purchased from Novus Biologicals). The formulation remained at room temperature for 1 week after mixing and were then dialyzed using 10 mM sodium phosphate buffer pH 7.4 and 10 kDa membrane molecular-weight cutoff Thermo Scientific™ Slide-A-Lyzer™ G2 dialysis cassettes for 48 h. As controls, infliximab was freshly prepared in buffer (positive control) and thermally-denatured by subjecting the infliximab in buffer to 90° C. for at least 20 min (negative control). Antibody concentrations were adjusted to 0.2 mg/mL using 10 mM sodium phosphate buffer pH 7.4 before analyzing secondary structure by circular dichroism (CD). Samples (400 μL) were loaded in rectangular quartz cells (1 mm path length, Starna Cells, 1-Q-1), and CD spectra in the far-UV region (195-260 nm) were collected using a Jasco J-815 CD spectropolarimeter.

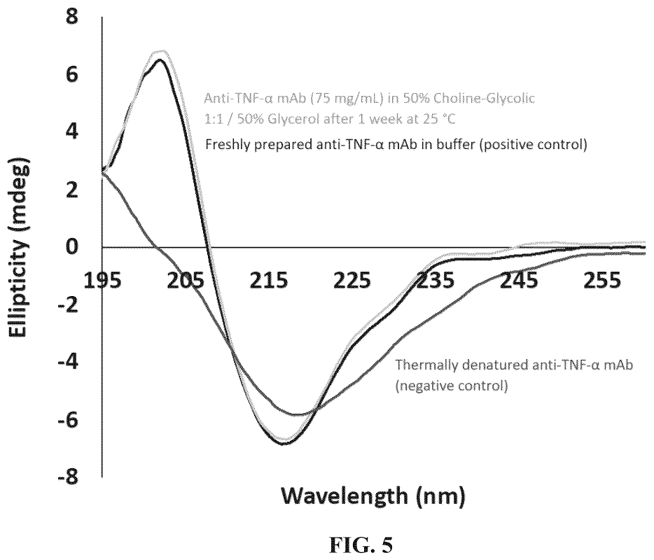

Anti-TNF-α antibody at 75 mg/mL in 50% Choline-Glycolic 1:1/50% Glycerol after 1 week at room temperature retained the same secondary structure by CD as compared to freshly prepared anti-TNF-α antibody in buffer, indicating the secondary structure of the antibody was not impacted by the presence of the ionic liquid, at this concentration, or under this storage condition, as shown in FIG. 5.

### Example 7: Stability of Infliximab in Choline-Glycolic by Differential Scanning Fluorimetry (DSF) After 1 Week at Room Temperature

A formulation containing 50% Choline-Glycolic 1:1 and 50% glycerol with final concentration 75 mg/mL of antibody was prepared (infliximab, a chimeric monoclonal anti-TNF-α antibody purchased from Novus Biologicals). The formulation remained at room temperature for 1 week after mixing and were then dialyzed using 10 mM sodium phosphate buffer pH 7.4 and 10 kDa membrane molecular-weight cutoff Thermo Scientific™ Slide-A-Lyzer™ G2 dialysis cassettes for 48 h. As controls, infliximab was freshly prepared in buffer (positive control). Antibody concentrations were adjusted to 0.2 mg/mL using 10 mM sodium phosphate buffer pH 7.4 before analyzing protein melting temperature (Tm) by differential scanning fluorimetry (DSF). SYPRO™ orange protein gel stain (5000×) was added to each sample to achieve a final concentration of 5× dye. Samples (20 μL) were loaded into a MicroAmp™ FAST optical 96-well reaction plate and covered with optical film. Using a modified QuantStudio 6/7 with decouple excitation and emission filters, samples were heated to 25° C. for 2 min at a rate of 1.6° C./s, ramped to 99° C. at a rate of 0.05° C./s, and held at 99° C. for 2 min. Detection was set using x1-m3 (excitation wavelength 470±15 nm and emission wavelength 586.5±10 nm). The peak of the first derivate of the thermal melting curve defined the melting temperature (Tm) for each sample. Data was analyzed Protein Thermal Shift™ Software v1.4.

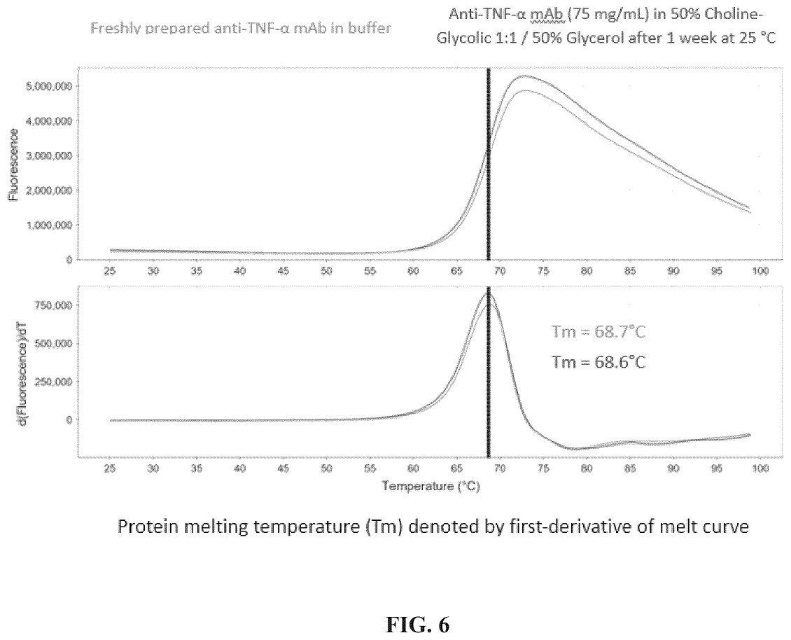

No shifts in protein melting temperature was observed between anti-TNF-α antibody (infliximab) at 75 mg/mL in 50% Choline-Glycolic 1:1/50% Glycerol after 1 week at room temperature (68.6° C.) compared to freshly prepared anti-TNF-α antibody in buffer (68.7° C.), indicating the stability of the antibody was not impacted by the presence of the ionic liquid, at this concentration, or under this storage condition, as shown in FIG. 6.

What is claimed is:

1. A composition comprising a therapeutic agent and at least one ionic liquid, wherein the therapeutic agent comprises an antibody or an antibody reagent.

2. The composition of claim 1, wherein the at least one ionic liquid comprises choline as a cation component.

3. The composition of claim 1 or 2, wherein the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

4. The composition of any one of claims 1-3, wherein the at least one ionic liquid comprises a cationic component and an anionic component in the molar ratio of 1;1, 1:2, 1:3, 1:4, 2:1, 3:1, or 4:1.

5. The composition of any one of claims 1-4, wherein the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

6. The composition of any one of claims 1-5, wherein the composition comprises a first ionic liquid and a second ionic liquid, wherein the first ionic liquid and the second ionic liquid are different.

7. The composition of claim 6, wherein the first ionic liquid comprises choline as a cation component, the second ionic liquid comprises choline as a cation component, or a combination thereof.

8. The composition of claim 6 or 7, wherein the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

9. The composition of any one of claims 6-8, wherein the first ionic liquid and the second ionic liquid comprise:
(i) choline-3-phenylpropanoic acid in the molar ratio of 1:2 and choline-glycolic acid in the molar ratio of 2:1, respectively;
(ii) choline-3-phenylpropanoic acid in the molar ratio of 1:1 and choline-glycolic acid in the molar ratio of 2:1, respectively; or
(iii) choline-3-phenylpropanoic acid in the molar ratio of 1:1 and choline-glycolic acid in the molar ratio of 1:1, respectively.

10. The composition of any one of claims 1-9, further comprising glycerol.

11. The composition of claim 10, wherein the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

12. The composition of claim 10 or 11, wherein the composition comprises at least one ionic liquid and glycerol in the ratio of:
(i) 10%:90%, wherein the at least one ionic liquid comprises choline-3-phenylpropanoic acid in the molar ratio of 1:1;
(ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1;
(iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1;
(iv) 75%:25%, wherein the at least one ionic liquid comprises choline-tartaric acid in the molar ratio of 2:1 or choline-citric acid in the molar ratio of 3:1; or
(v) 90%:10%, wherein the at least one ionic liquid comprises choline-tartaric acid in the molar ratio of 2:1.

13. The composition of claim 10 or 11, wherein the composition comprises the first ionic liquid and the second ionic liquid, and wherein the composition comprises the first ionic liquid, the second ionic liquid, and glycerol in the ratio of:
(i) 10%:45%:45%,
wherein the first ionic liquid comprises choline-3-phenylpropanoic acid in the molar ratio of 1:2 and the second ionic liquid comprises choline-glycolic acid in the molar ratio of 2:1; or
wherein the first ionic liquid comprises choline-3-phenylpropanoic acid in the molar ratio of 1:1 and the second ionic liquid comprises choline-glycolic acid in the molar ratio of 2:1; or
(ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

14. The composition of any one of claims 1-13, wherein the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab-adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

15. The composition of any one of claims 1-13, wherein the antibody or the antibody reagent is infliximab or a biosimilar thereof, adalimumab or a biosimilar thereof, or a combination thereof.

16. The composition of claim 15, wherein the infliximab or a biosimilar thereof is selected from the group consisting of infliximab, infliximab-dyyb, infliximab-abda, infliximab-qbtx, and infliximab-axxq.

17. The composition of claim 15 or 16, wherein the adalimumab or a biosimilar thereof is selected from the group consisting of adalimumab, adalimumab-atto, adalimumab-adaz, adalimumab-afzb, and adalimumab-bwwd.

18. The composition of any one of claims 1-17, further comprising one or more additional agents.

19. The composition of claim 18, wherein the one or more additional agents are selected from the group consisting of a nucleic acid, a small molecule, and a polypeptide.

20. The composition of claim 18 or 19, wherein the one or more additional agents are selected from the group consisting of a corticosteroid, an aminosalicylates, an immunomodulator, a monoclonal antibody, and a biologic.

21. The composition of any one of claims 1-20, wherein the composition is formulated for delivery to a mucus membrane, across a mucus membrane, or a combination thereof.

22. The composition of claim 21, wherein the mucus membrane is a nasal membrane, an oral membrane, a vaginal membrane, or a combination thereof.

23. The composition of any one of claims 1-22, wherein the composition is formulated for administered to a gastrointestinal tract.

24. The composition of any one of claims 1-23, wherein the composition is formulated for subcutaneous administration, intravenous administration, topical administration, intrajejunal administration, oral administration, or a combination thereof.

25. The composition of any one of claims 1-24, wherein the composition is formulated for oral administration.

26. The composition of any one of claims 1-25, wherein the composition is formulated in a form selected from the group consisting of a tablet, a pill, a caplet, a capsule, a spray, an aerosol, a syrup, a liquid, and a combination thereof.

27. The composition of any one of claims 1-26, wherein the composition is encapsulated in a capsule.

28. A pharmaceutical composition comprising the composition of any one of claims 1-27, and a pharmaceutically acceptable excipient.

29. A method of treating a disease or a disorder in a subject in need thereof, comprising administering to the subject a therapeutically effective amount of the composition of any one of claims 1-27 or the pharmaceutical composition of claim 28.

30. The method of claim 29, wherein the disease or the disorder is an inflammatory disease, an autoimmune disease, or a combination thereof.

31. The method of claim 30, wherein the inflammatory disease, the autoimmune disease, or a combination thereof is characterized by a hyperactive immune system.

32. The method of claim 30 or 31, wherein the inflammatory disease, the autoimmune disease, or a combination thereof is selected from the group consisting of rheumatoid arthritis, Crohn's disease, ulcerative colitis, psoriasis, psoriatic arthritis, ankylosing spondylitis, inflammatory bowel disease, and a combination thereof.

33. The method of claim 29, wherein the disease or the disorder is diabetes.

34. The method of any one of claims 29-33, further comprising administering one or more additional agents.

35. The method of claim 34, wherein the one or more additional agents are selected from the group consisting of a nucleic acid, a small molecule, and a polypeptide.

36. The method of claim 34, wherein the one or more additional agents are selected from the group consisting of a corticosteroid, an aminosalicylates, an immunomodulator, a monoclonal antibody, and a biologic.

37. The method of any one of claims 29-36, wherein the method delivers the composition to a mucus membrane, across a mucus membrane, or a combination thereof in the subject.

38. The method of claim 37, wherein the mucus membrane is a nasal membrane, an oral membrane, a vaginal membrane, or a combination thereof.

39. The method of any one of claims 29-38, wherein the administering comprises administering to a gastrointestinal tract in the subject.

40. The method of any one of claims 29-39, wherein the administering comprises subcutaneous administration, intravenous administration, topical administration, intrajejunal administration, oral administration, or a combination thereof.

41. The method of any one of claims 29-40, wherein the administering comprises oral administration.

42. The method of any one of claims 29-41, wherein the composition is administered in a single dose.

43. The method of any one of claims 29-41, wherein the composition is administered in multiple doses.

44. A method of increasing the solubility of a therapeutic agent comprising formulating a composition comprising the therapeutic agent and at least one ionic liquid,
wherein the therapeutic agent comprises an antibody or an antibody reagent.

45. The method of claim 44, wherein the at least one ionic liquid comprises choline as a cation component.

46. The method of claim 44 or 45, wherein the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

47. The method of any one of claims 44-46, wherein the at least one ionic liquid comprises a cationic component and an anionic component in the molar ratio of 1;1, 1:2, 1:3, 1:4, 2:1, 3:1, or 4:1.

48. The method of any one of claims 44-47, wherein the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

49. The method of any one of claims 44-48, wherein the composition comprises a first ionic liquid and a second ionic liquid, wherein the first ionic liquid and the second ionic liquid are different.

50. The method of claim 49, wherein the first ionic liquid comprises choline as a cation component, the second ionic liquid comprises choline as a cation component, or a combination thereof.

51. The method of claim 49 or 50, wherein the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

52. The method of any one of claims 49-51, wherein the first ionic liquid and the second ionic liquid comprise:
(i) choline-3-phenylpropanoic acid in the molar ratio of 1:2 and choline-glycolic acid in the molar ratio of 2:1, respectively;
(ii) choline-3-phenylpropanoic acid in the molar ratio of 1:1 and choline-glycolic acid in the molar ratio of 2:1, respectively; or
(iii) choline-3-phenylpropanoic acid in the molar ratio of 1:1 and choline-glycolic acid in the molar ratio of 1:1, respectively.

53. The method of any one of claims 44-52, further comprising glycerol.

54. The method of claim 53, wherein the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

55. The method of claim 53 or 54, wherein the composition comprises at least one ionic liquid and glycerol in the ratio of:
(i) 10%:90%, wherein the at least one ionic liquid comprises choline-3-phenylpropanoic acid in the molar ratio of 1:1;
(ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1;
(iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1;
(iv) 75%:25%, wherein the at least one ionic liquid comprises choline-tartaric acid in the molar ratio of 2:1 or choline-citric acid in the molar ratio of 3:1; or
(v) 90%:10%, wherein the at least one ionic liquid comprises choline-tartaric acid in the molar ratio of 2:1.

56. The method of claim 53 or 54, wherein the composition comprises the first ionic liquid and the second ionic liquid, and wherein the composition comprises the first ionic liquid, the second ionic liquid, and glycerol in the ratio of:
(i) 10%:45%:45%,
wherein the first ionic liquid comprises choline-3-phenylpropanoic acid in the molar ratio of 1:2 and the second ionic liquid comprises choline-glycolic acid in the molar ratio of 2:1; or
wherein the first ionic liquid comprises choline-3-phenylpropanoic acid in the molar ratio of 1:1 and the second ionic liquid comprises choline-glycolic acid in the molar ratio of 2:1; or
(ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

57. The method of any one of claims 44-56, wherein the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab-adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

58. The method of any one of claims 44-56, wherein the antibody or the antibody reagent is infliximab or a biosimilar thereof, adalimumab or a biosimilar thereof, or a combination thereof.

59. The method of claim 58, wherein the infliximab or a biosimilar thereof is selected from the group consisting of infliximab, infliximab-dyyb, infliximab-abda, infliximab-qbtx, and infliximab-axxq.

60. The method of claim 58 or 59, wherein the adalimumab or a biosimilar thereof is selected from the group consisting of adalimumab, adalimumab-atto, adalimumab-adaz, adalimumab-afzb, and adalimumab-bwwd.

61. The method of any one of claims 44-60, wherein the composition further comprises one or more additional agents.

62. The method of claim 61, wherein the one or more additional agents are selected from the group consisting of a nucleic acid, a small molecule, and a polypeptide.

63. The method of claim 61 or 62, wherein the one or more additional agents are selected from the group consisting of a corticosteroid, an aminosalicylates, an immunomodulator, a monoclonal antibody, and a biologic.

64. The method of any one of claims 44-63, wherein the composition is formulated for delivery to a mucus membrane, across a mucus membrane, or a combination thereof.

65. The method of claim 64, wherein the mucus membrane is a nasal membrane, an oral membrane, a vaginal membrane, or a combination thereof.

66. The method of any one of claims 44-65, wherein the composition is formulated for administered to a gastrointestinal tract.

67. The method of any one of claims 44-66, wherein the composition is formulated for subcutaneous administration, intravenous administration, topical administration, intrajejunal administration, oral administration, or a combination thereof.

68. The method of any one of claims 44-67, wherein the composition is formulated for oral administration.

69. The method of any one of claims 44-68, wherein the composition is formulated in a form selected from the group consisting of a tablet, a pill, a caplet, a capsule, a spray, an aerosol, a syrup, a liquid, and a combination thereof.

70. The method of any one of claims 44-69, wherein the composition is encapsulated in a capsule.

71. A method of enhancing delivery of a therapeutic agent into a systemic circulation in a subject in need thereof comprising administering a composition comprising the therapeutic agent and at least one ionic liquid to the subject,
wherein the therapeutic agent comprises an antibody or an antibody reagent.

72. The method of claim 71, wherein the at least one ionic liquid comprises choline as a cation component.

73. The method of claim 71 or 72, wherein the at least one ionic liquid comprises an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

74. The method of any one of claims 71-73, wherein the at least one ionic liquid comprises a cationic component and an anionic component in the molar ratio of 1;1, 1:2, 1:3, 1:4, 2:1, 3:1, or 4:1.

75. The method of any one of claims 71-74, wherein the at least one ionic liquid comprises choline-3-phenylpropanoic acid in the molar ratio of 1:1, choline-glycolic acid in the molar ratio of 1:1, choline-glycolic acid in the molar ratio of 2:1, choline-glycolic acid in the molar ratio of 1:2, choline-malic acid in the molar ratio of 2:1, choline-malic acid in the molar ratio of 1:1, choline-tartaric acid in the molar ratio of 2:1, choline-lactic acid in the molar ratio of 1:1, choline-cinnamic acid in the molar ratio of 1:1, choline-citric acid in the molar ratio of 3:1, choline-succinic acid in the molar ratio of 2:1, or any combination thereof.

76. The method of any one of claims 71-75, wherein the composition comprises a first ionic liquid and a second ionic liquid, wherein the first ionic liquid and the second ionic liquid are different.

77. The method of claim 76, wherein the first ionic liquid comprises choline as a cation component, the second ionic liquid comprises choline as a cation component, or a combination thereof.

78. The method of claim 76 or 77, wherein the first ionic liquid and the second ionic liquid independently comprise an anionic component selected from the group consisting of malonic acid, 3-phenylpropanoic acid, mandelic acid, DL-2-phenylpropionic acid, glycolic acid, malic acid, tartaric acid, 3-(4-hydroxyphenyl)propionic acid, decanoic acid, lactic acid, cinnamic acid, acetic acid, citric acid, phosphoric acid, 2-octenoic acid, citronellic acid, succinic acid, salicylic acid, and benzoic acid.

79. The method of any one of claims 76-78, wherein the first ionic liquid and the second ionic liquid comprise:
(i) choline-3-phenylpropanoic acid in the molar ratio of 1:2 and choline-glycolic acid in the molar ratio of 2:1, respectively;
(ii) choline-3-phenylpropanoic acid in the molar ratio of 1:1 and choline-glycolic acid in the molar ratio of 2:1, respectively; or
(iii) choline-3-phenylpropanoic acid in the molar ratio of 1:1 and choline-glycolic acid in the molar ratio of 1:1, respectively.

80. The method of any one of claims 71-79, further comprising glycerol.

81. The method of claim 80, wherein the composition comprises at least one ionic liquid and glycerol in the ratio of 95%:5%, 90%:10%, 85%:15%, 80%:20%, 75%:25%, 70%:30, 65%:35%, 60%:40%, 55%:45%, 50%:50%, 45%:55%, 40%:60%, 35%:65%, 30%:70%, 25%:75%, 20%:80%, 15%:85%, 10%:90%, or 5%:95%.

82. The method of claim 80 or 81, wherein the composition comprises at least one ionic liquid and glycerol in the ratio of:
(i) 10%:90%, wherein the at least one ionic liquid comprises choline-3-phenylpropanoic acid in the molar ratio of 1:1;
(ii) 25%:75%, wherein the at least one ionic liquid comprises choline-3-phenylpropanoic acid in the molar ratio of 1:1, choline-glycolic acid in the molar ratio of 1:2, or choline-cinnamic acid in the molar ratio of 1:1;
(iii) 50%:50%, wherein the at least one ionic liquid comprises choline-glycolic acid in the molar ratio of 1:1, 2:1, or 1:2, choline-malic acid in the molar ratio of 2:1 or 1:1, choline-tartaric acid in the molar ratio of 2:1, choline-lactic acid in the molar ratio of 1:1, or choline-succinic acid in the molar ratio of 2:1;
(iv) 75%:25%, wherein the at least one ionic liquid comprises choline-tartaric acid in the molar ratio of 2:1 or choline-citric acid in the molar ratio of 3:1; or
(v) 90%:10%, wherein the at least one ionic liquid comprises choline-tartaric acid in the molar ratio of 2:1.

83. The method of claim 80 or 81, wherein the composition comprises the first ionic liquid and the second ionic liquid, and wherein the composition comprises the first ionic liquid, the second ionic liquid, and glycerol in the ratio of:
(i) 10%:45%:45%,
wherein the first ionic liquid comprises choline-3-phenylpropanoic acid in the molar ratio of 1:2 and the second ionic liquid comprises choline-glycolic acid in the molar ratio of 2:1; or
wherein the first ionic liquid comprises choline-3-phenylpropanoic acid in the molar ratio of 1:1 and the second ionic liquid comprises choline-glycolic acid in the molar ratio of 2:1; or
(ii) 10%:40%:50%, wherein the first ionic liquid comprises choline-3-phenylpropanoic acid in the molar ratio of 1:1 and the second ionic liquid comprises choline-glycolic acid in the molar ratio of 1:1.

84. The method of any one of claims 71-83, wherein the antibody or the antibody reagent is selected from the group consisting of abciximab, adalimumab, adlimumab-atto, ado-trastuzumab, ado-trastuzumab emtansine, alemtuzumab, alirocumab, atezolizumab, avelumab, basiliximab, belimumab, bevacizumab, bezlotoxumab, blinatumomab, brentuximab, brentuximab vedotin, brodalumab, canakinumab, capromab, capromab pendetide, certolizumab, certolizumab pegol, cetuximab, daclizumab, daratumumab, denosumab, dinutuximab, dupilumab, durvalumab, eculizumab, elotuzumab, evolocumab, etanercept, etanercept-szzs, golimumab, ibritumomab, ibritumomab tiuxetan, idarucizumab, infliximab, infliximab-abda, infliximab-dyyb, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucriumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, inotuzumab, adalimumab-adbm, gemtuzumab ozogamicin, gemtuzumab, bevacizumab-awwb, benralizumab, emicizumab, emicizumab-kxwh, trastuzumab-dkst, infliximab-qbtx, ibalizumab, ibalizumab-uiyk, tildrakizumab, tildrakizumab-asmn, burosumab, burosumab-twza, erenumab, erenumab-aooe, tositumomab, mogamulizumab, moxetumomab, moxetumomab pasudotox, cemiplimab, polatuzumab, catumaxomab, polatuzumab vedotin, and any combinations thereof.

85. The method of any one of claims 71-83, wherein the antibody or the antibody reagent is infliximab or a biosimilar thereof, adalimumab or a biosimilar thereof, or a combination thereof.

86. The method of claim 85, wherein the infliximab or a biosimilar thereof is selected from the group consisting of infliximab, infliximab-dyyb, infliximab-abda, infliximab-qbtx, and infliximab-axxq.

87. The method of claim 85 or 86, wherein the adalimumab or a biosimilar thereof is selected from the group consisting of adalimumab, adalimumab-atto, adalimumab-adaz, adalimumab-afzb, and adalimumab-bwwd.

88. The method of any one of claims 71-87, the composition further comprises one or more additional agents.

89. The method of claim 88, wherein the one or more additional agents are selected from the group consisting of a nucleic acid, a small molecule, and a polypeptide.

90. The method of claim 88 or 89, wherein the one or more additional agents are selected from the group consisting of a corticosteroid, an aminosalicylates, an immunomodulator, a monoclonal antibody, and a biologic.

91. The method of any one of claims 71-90, wherein the composition is formulated for delivery to a mucus membrane, across a mucus membrane, or a combination thereof.

92. The method of claim 91, wherein the mucus membrane is a nasal membrane, an oral membrane, a vaginal membrane, or a combination thereof.

93. The method of any one of claims 71-92, wherein the composition is formulated for administered to a gastrointestinal tract.

94. The method of any one of claims 71-93, wherein the composition is formulated for subcutaneous administration, intravenous administration, topical administration, intrajejunal administration, oral administration, or a combination thereof.

95. The method of any one of claims 71-94, wherein the composition is formulated for oral administration.

96. The method of any one of claims 71-95, wherein the composition is formulated in a form selected from the group consisting of a tablet, a pill, a caplet, a capsule, a spray, an aerosol, a syrup, a liquid, and a combination thereof.

97. The method of any one of claims 71-96, wherein the composition is encapsulated in a capsule.

98. The method of any one of claims 71-97, wherein the subject has a disease or a disorder,
wherein the disease or the disorder is an inflammatory disease, an autoimmune disease, or a combination thereof.

99. The method of claim 98, wherein the inflammatory disease, the autoimmune disease, or a combination thereof is characterized by a hyperactive immune system.

100. The method of claim 98 or 99, wherein the inflammatory disease, the autoimmune disease, or a combination thereof is selected from the group consisting of rheumatoid arthritis, Crohn's disease, ulcerative colitis, psoriasis, psoriatic arthritis, ankylosing spondylitis, inflammatory bowel disease, and a combination thereof.

101. The method of any one of claims 71-97, wherein the subject has a disease or a disorder,
wherein the disease or the disorder is diabetes.

102. The method of any one of claims 71-101, wherein the method delivers the composition to a mucus membrane, across a mucus membrane, or a combination thereof in the subject.

103. The method of claim 102, wherein the mucus membrane is a nasal membrane, an oral membrane, a vaginal membrane, or a combination thereof.

104. The method of any one of claims 71-103, wherein the administering comprises administering to a gastrointestinal tract in the subject.

105. The method of any one of claims 71-104, wherein the administering comprises subcutaneous administration, intravenous administration, topical administration, intrajejunal administration, oral administration, or a combination thereof.

106. The method of any one of claims 71-105, wherein the administering comprises oral administration.

107. The method of any one of claims 71-106, wherein the composition is administered in a single dose.

108. The method of any one of claims 71-107, wherein the composition is administered in multiple doses.
